(19) United States
(12) Patent Application Publication
(10) Pub. No.: US 2010/0022778 A1
(43) Pub. Date: Jan. 28, 2010

(54) PROCESS FOR PRODUCTION OF SULFINYLBENZIMIDAZOLE COMPOUNDS OR SALTS THEREOF

(76) Inventors: Atsushi Kamada, Ibaraki (JP); Daisuke Iida, Ibaraki (JP); Hiroki Terauchi, Ibaraki (JP)

Correspondence Address: BIRCH STEWART KOLASCH & BIRCH, PO BOX 747, FALLS CHURCH, VA, 22040-0747, US

(21) Appl. No.: 12/441,075

(22) PCT Filed: Oct. 11, 2007
(86) PCT No.: PCT/JP2007/070302

§ 371 Date: Mar. 12, 2009

(30) Foreign Application Priority Data
Oct. 13, 2006 (JP) 2006-279581

(51) Int. Cl. C07D 405/14

(52) U.S. Cl. 546/273.7

(57) ABSTRACT

It is an object of the present invention to provide a process for the production on an industrial scale of a compound (1) or a salt thereof. There is provided a process for the production of compound (1) represented by the formula or a salt thereof: (wherein R1 and R3 independently represents a hydrogen atom or a methyl group), the process comprising the steps of: (a) reacting together a compound (3T) represented by the formula: (wherein X1 represents a leaving group, and R1 and R3 are defined as above) and (2,2-dimethyl-1,3-dioxan-5-yl)methanol represented by the formula or a salt thereof: so as to produce a compound (2T) represented by the formula (wherein R1 and R3 are defined as above); and (b) reacting the compound represented by above formula (2T) with an oxidizing agent.

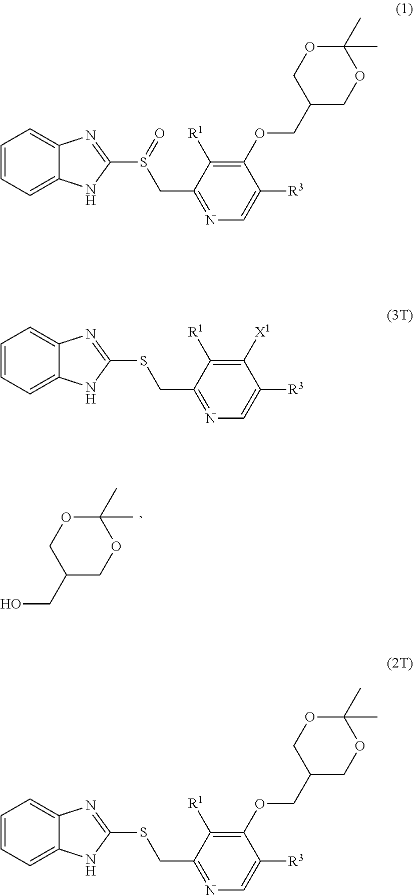

### TECHNICAL FIELD

The present invention relates to a process for the production of benzimidazole compounds or salts thereof that are useful as a gastric acid secretion inhibiting agent.

### BACKGROUND ART

It is thought that peptic ulcers such as gastric ulcers and duodenal ulcers occur as a result of autodigestion brought about when the balance between attacking factors such as an acid and a pepsin and defensive factors such as mucus and a blood flow is destroyed.

Treatment of peptic ulcers is carried out through internal medicine as a rule, and various drug treatments have been tried. In particular, drugs such as omeprazole, esomeprazole, pantoprazole, lansoprazole and rabeprazole that specifically inhibit H+, K+-ATPase which is an enzyme that is present in gastric parietal cells and governs the final process of gastric acid secretion, so as to suppress acid secretion, and as a result prevent autodigestion have recently been developed and put to clinical use.

These drugs have an excellent therapeutic effect, but there are still demands for a drug that has yet better persistence of the gastric acid secretion inhibiting action, is safer, and has suitable physiochemical stability.

### DISCLOSURE OF INVENTION

### Problems to be Solved by the Invention

At the time of filing the present application, it had not been disclosed that 2-[[[4-[(2,2-dimethyl-1,3-dioxan-5-yl)methoxy]-3,5-dimethylpyridin-2-yl]methyl]sulfinyl]-1H-benzimidazole (hereinafter referred to as “compound (1)”) or a salt thereof exhibits an excellent gastric acid secretion inhibiting action (in particular has good persistence of the gastric acid secretion inhibiting action, it being possible to keep the pH in the stomach high for a prolonged period of time), and moreover is useful as a drug for treating or preventing reflux esophagitis, symptomatic reflux esophagitis, and gastric and duodenal ulcers.

There has thus also been no disclosure of a process for the production of 2-[[[4-[(2,2-dimethyl-1,3-dioxan-5-yl)methoxy]-3,5-dimethylpyridin-2-yl]methyl]sulfinyl]-1H-benzimidazole or a salt thereof.

It is an object of the present invention to provide a process for the production of compound (1) or a salt thereof which is useful as a compound having an excellent gastric acid secretion inhibiting action.

### Means for Solving the Problems

The present inventors carried out studies with vigor, and as a result, completed the present invention upon discovering a useful process for the production of compound (1) or a salt thereof.

That is, the present invention provides:

[1] a process for the production of a compound (1) represented by the formula or a salt thereof

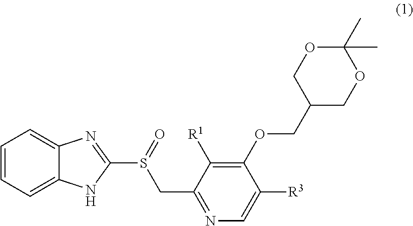

(wherein R1 and R3 independently represent a hydrogen atom or a methyl group), the process comprising the steps of:

(a) reacting together a compound (3T) represented by the formula

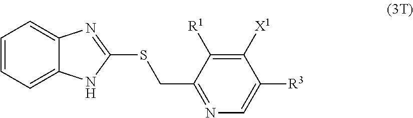

(wherein X1 represents a leaving group, and R1 and R3 are defined as above) and (2,2-dimethyl-1,3-dioxan-5-yl)methanol represented by the formula or a hydrate thereof,

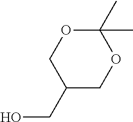

so as to produce a compound (2T) represented by the formula

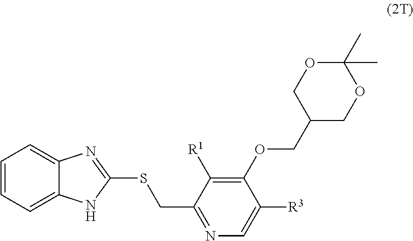

(wherein R1 and R3 are defined as above); and

(b) reacting the compound represented by the formula (2T) with an oxidizing agent;

[2] a process for the production of a compound (1) represented by the formula or a salt thereof

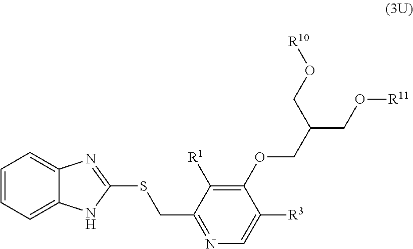

(wherein R1 and R3 independently represent a hydrogen atom or a methyl group), the process comprising the steps of:

(c) subjecting a compound (3U) represented by the formula

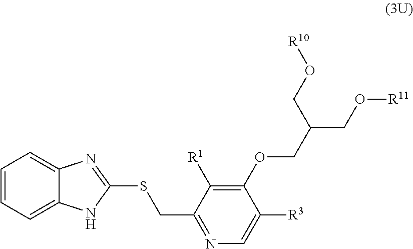

(wherein R10 and R11 independently represent a hydrogen atom or a protecting group of a hydroxyl group, or R10 and R11 are combined so as to represent a methylene group (the methylene group optionally having thereon one or two groups selected from a C1-C6 alkyl group, a C1-C6 alkoxy group, a phenyl group optionally having thereon one or two methoxy groups, and trichloromethyl groups), a carbonyl group, a 1,1-cyclopropylene group, a 1,1-cyclobutylene group, a 1,1-cyclopentylene group, or a 1,1-cyclohexylene group (provided that R10 and R11 are combined so as to represent a 2,2-propylene group being excluded), and R1 and R3 are defined as above)
to a deprotection reaction (provided that when both R10 and R11 represent hydrogen atom, this deprotection reaction is not required), and then reacting with an acetalizing reagent, so as to produce a compound (2T) represented by the formula

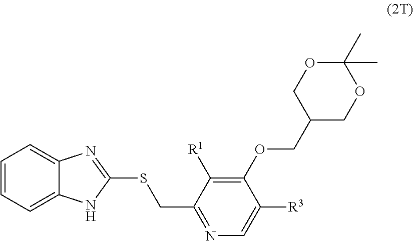

(wherein R1 and R3 are defined as above); and

(b) reacting the compound represented by the formula (2T) with an oxidizing agent;

[3] a process for the production of a compound (1Z) represented by the formula or a salt thereof

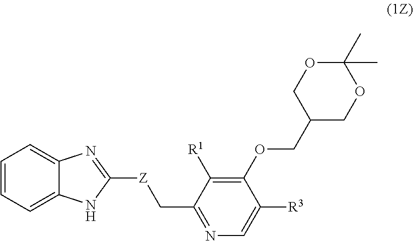

(wherein Z represents —S— or —SO—, and R1 and R3 independently represent a hydrogen atom or a methyl group),
the process comprising:

reacting together a compound (2Z) represented by the formula

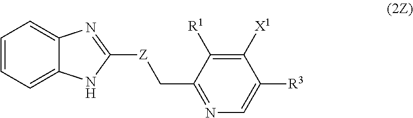

(wherein X1 represents a leaving group, and Z, R1 and R3 are defined as above) and (2,2-dimethyl-1,3-dioxan-5-yl)methanol represented by the formula

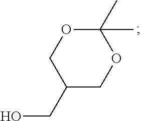

[4] a process for the production of a compound (1Z) represented by the formula or a salt thereof

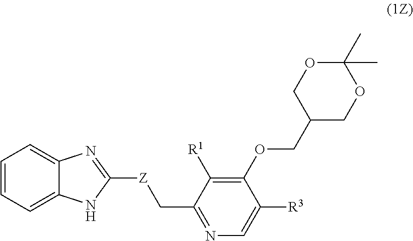

(wherein Z, R1 and R3 are defined as above), the process comprising:

subjecting a compound (3Z) represented by the formula

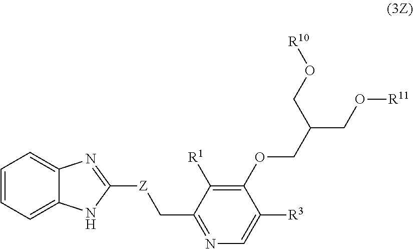

(wherein Z represents —S— or —SO—, and R10 and R11 independently represent a hydrogen atom or a protecting group of a hydroxyl group, or R10 and R11 are combined so as to represent a methylene group (the methylene group optionally having thereon one or two groups selected from a C1-C6 alkyl group, a C1-C6 alkoxy group, a phenyl group optionally having thereon one or two methoxy groups, and trichloromethyl groups), a carbonyl group, a 1,1-cyclopropylene group, a 1,1-cyclobutylene group, a 1,1-cyclopentylene group, or a 1,1-cyclohexylene group (provided that R10 and R11 are combined so as to represent a 2,2-propylene group being excluded))
to a deprotection reaction,

and then reacting with an acetalizing reagent.

Following is a more detailed description of the present invention, with the meanings of terms and symbols used in the present specification being explained.

In the present specification, a salt of compound (1) is a salt formed by the NH group in the 1- or 3-position of the benzimidazole skeleton.

There are no particular limitations on such a salt so long as the salt is pharmacologically acceptable. Examples of the salt include inorganic base salts and organic base salts, with the inorganic base salts being preferable.

Preferable examples of such the inorganic base salt include a sodium salt, a calcium salt, and a magnesium salt.

In the present specification, it may that the structural formula of compound (1) shows one particular isomer for convenience; however, all optical isomers that arise for the structure of compound (1) and also mixtures of such isomers are included, and there is no limitation to that shown in the formula for convenience, it being possible for the compound to be any of the isomers or a mixture thereof. Accordingly, for compound (1), an optically active substance or a racemate may exist, and in the present specification there is no limitation to one of these, with all being included.

In the present specification, R1 and R3 independently represent a hydrogen atom or a methyl group, each of R1 and R3 preferably being a methyl group.

In the present specification, R10 and R11 independently represent a hydrogen atom or a protecting group of a hydroxyl group, or R10 and R11 are combined so as to represent a methylene group (the methylene group optionally having thereon one or two groups selected from C1-C6 alkyl groups, C1-C6 alkoxy groups, phenyl groups optionally having thereon one or two methoxy groups, and trichloromethyl groups), a carbonyl group, a 1,1-cyclopropylene group, a 1,1-cyclobutylene group, a 1,1-cyclopentylene group, or a 1,1-cyclohexylene group (provided that R10 and R11 are combined so as to represent a 2,2-propylene group being excluded); R10 and R11 are preferably combined so as to represent a 1,1-cyclobutylene group, a 1,1-cyclopentylene group, or a 1,1-cyclohexylene group.

In the present specification, Z represents —S— or —SO—, preferably —S—.

In the present specification, X1 represents a leaving group. Specific examples of the leaving group include sulfonyloxy groups such as methanesulfonyloxy, p-toluenesulfonyloxy and trifluoromethanesulfonyloxy, halogen atoms such as chlorine, bromine and iodine, acyloxy groups such as acetyloxy, trifluoroacetyloxy and propionyloxy, sulfonyl groups such as benzenesulfonyl; and a nitro group; a chlorine atom, a bromine atom, an iodine atom, or a nitro group is preferable, a chlorine atom being particularly preferable.

In the present specification, an acetalizing reagent refers to a reagent for forming an isopropylidene ketal (—CH(CH₃)₂—). Specific examples of the acetalizing agent include (1) acetone, (2) compounds represented by the formula (CH₃)₂C(OR^{1X})₂ (wherein R^{1X} represents a C1-C6 alkyl group or a trimethylsilyl group), and (3) compounds represented by the formula (CH₃)C(OR^{1X})=CH₂ (wherein R^{1X} represents a C1-C6 alkyl group or a trimethylsilyl group), with acetone, 2,2-diethoxypropane, or 2-methoxypropene being preferable.

In the present specification, examples of an oxidizing agent include hydrogen peroxide, t-butyl hydroperoxide, cumene hydroperoxide, sodium periodate, peracetic acid, perbenzoic acid, 3-chloroperbenzoic acid, and a urea hydrogen peroxide adduct ((NH₂)₂CO.H₂O₂); 3-chloroperbenzoic acid or cumene hydroperoxide is preferable, cumene hydroperoxide being particularly preferable.

In the present specification, there are no particular limitations on a protecting group of a hydroxyl group so long as this is a group that can be used as a protecting group for a hydroxyl group. Specific examples of the protecting group include C1-C6 acyl groups (an acetyl group etc.), a tetrahydropyranyl group, a trimethylsilyl group, a t-butyldimethylsilyl group, and a benzyl group (the benzyl group may have a substituent such as a methoxy group or a nitro group).

In the present specification, there are no particular limitations on the deprotection reaction so long as the reaction conditions are ones generally used for eliminating both R10 and R11 so as to convert each of R10 and R11 into a hydrogen atom. Preferable examples of the deprotection reaction include (1) in the presence of an acid, (2) in the presence of a base, (3) in the presence of a fluorine reagent (e.g. tetrabutylammonium fluoride), and (4) in the presence of a reducing reagent. There can be used reaction conditions for elimination (reaction conditions for eliminating protecting groups) generally used in organic synthesis, this being in accordance with R10 and R11 as appropriate.

### ADVANTAGEOUS EFFECTS OF THE INVENTION

The process according to the present invention can be used in the production on an industrial scale of a compound (1) or a salt thereof which is useful as a compound having an excellent gastric acid secretion inhibiting action.

### BEST MODE FOR CARRYING OUT THE INVENTION

In the following, processes according to the present invention for producing a compound (1) or a salt thereof including an etherification reaction or an acetalization reaction are described. The compound (1) or the salt thereof can be produced using a method P or method Q described below.

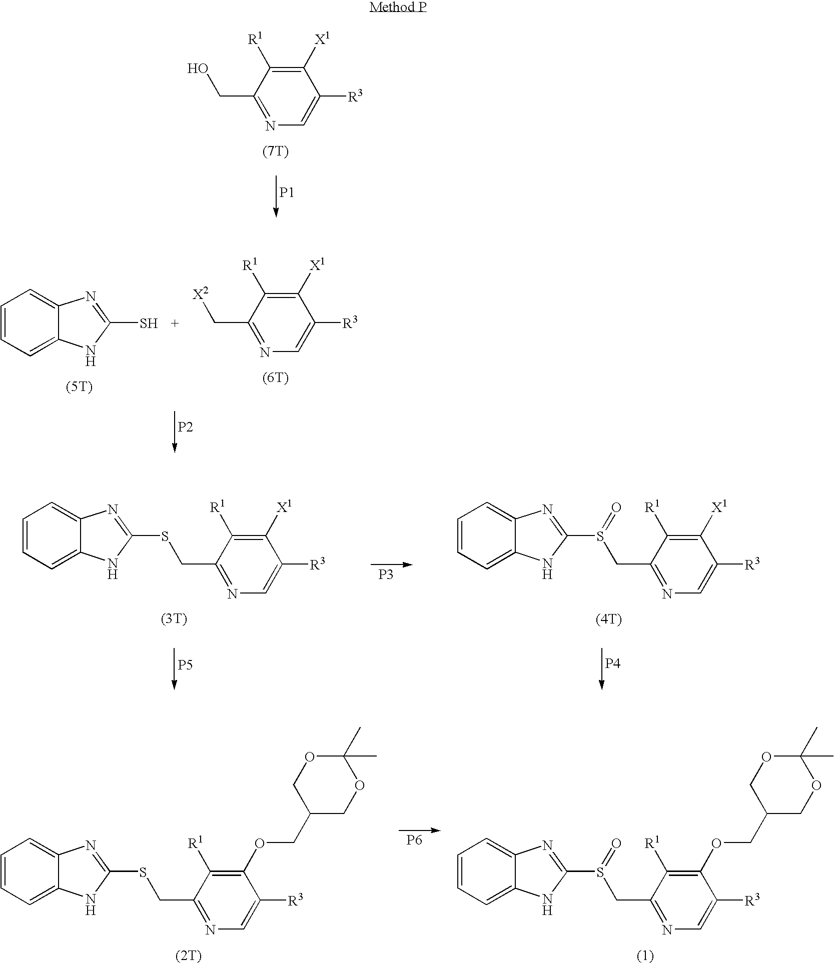

In the above method P, R1 and R3 are defined as above, and X2 represents a leaving group.

Examples of the leaving group X2 include sulfonyloxy groups such as methanesulfonyloxy, p-toluenesulfonyloxy and trifluoromethanesulfonyloxy; halogen groups such as chlorine, bromine and iodine; and acyloxy groups such as acetyloxy, trifluoroacetyloxy and propionyloxy; methanesulfonyloxy, p-toluenesulfonyloxy, chlorine, or an acetyloxy group is preferable.

X1 represents a leaving group. Specific examples of the leaving group include sulfonyloxy groups such as methanesulfonyloxy, p-toluenesulfonyloxy and trifluoromethanesulfonyloxy; halogen groups such as chlorine, bromine and iodine; acyloxy groups such as acetyloxy, trifluoroacetyloxy and propionyloxy; sulfonyl groups such as benzenesulfonyl; and a nitro group; a chlorine atom, a bromine atom, an iodine atom, or a nitro group is preferable, a chlorine atom being particularly preferable.

Following is a description of each of the steps in the method P.

### Step P1: Introduction of Leaving Group or Halogenation

### (1) Leaving Group Introduction Reaction

The present step is a step of reacting compound (7T) and a leaving group introducing agent together in the presence of a base either without a solvent or in an inert solvent, so as to produce compound (6T) or a salt thereof.

There are no particular limitations on a solvent used so long as this is a solvent in which the starting material dissolves to some extent and that does not impede the reaction. Examples of the solvent include halogenated hydrocarbons such as chloroform, dichloromethane, 1,2-dichloroethane or carbon tetrachloride; aromatic hydrocarbons such as benzene, toluene or benzotrifluoride; ethers such as diethyl ether, diisopropyl ether, tetrahydrofuran, dioxane, dimethoxyethane or diethylene glycol dimethyl ether; amides such as formamide, N,N-dimethylformamide, N,N-dimethylacetamide or hexamethylphosphoric acid triamide, or pyridine; or a mixed solvent thereof; halogenated hydrocarbons, ethers, or a mixed solvent of ethers and aromatic hydrocarbons is preferable, dichloromethane, tetrahydrofuran, or a mixed solvent of tetrahydrofuran and toluene being most preferable.

Examples of the leaving group introducing agent used include sulfonylating agents such as methanesulfonyl chloride, p-toluenesulfonyl chloride, trifluoromethanesulfonyl chloride and N-phenyl-bis(trifluoromethanesulfonimide); methanesulfonyl chloride or p-toluenesulfonyl chloride is preferable, methanesulfonyl chloride being most preferable.

Examples of the base used include tertiary alkyl amines such as trimethylamine and triethylamine; pyridine; potassium carbonate; sodium carbonate, sodium hydroxide; and potassium hydroxide; triethylamine or sodium hydroxide is preferable, triethylamine being most preferable.

The reaction temperature depends on the starting material, the solvent, the leaving group introducing agent, and the base, but is generally in a range of from −50 to 100° C., preferably from −20 to 40° C.

The reaction time depends on the starting material, the solvent, the leaving group introducing agent, the base, and the reaction temperature, but is generally in a range of from 15 minutes to 12 hours, preferably from 30 minutes to 2 hours.

The compound obtained in the present step can be used as is in the next step without being isolated in particular.

### (2) Halogenation Reaction (Taking a Chlorination Reaction as a Representative Example)

The present step is a step of reacting compound (7T) with a chlorinating agent either in the presence of or without a base and either without a solvent or in an inert solvent, so as to produce compound (6T).

There are no particular limitations on a solvent used so long as this is a solvent in which the starting material dissolves to some extent and that does not impede the reaction. Examples of the solvent include halogenated hydrocarbons such as chloroform, dichloromethane, 1,2-dichloroethane or carbon tetrachloride; aromatic hydrocarbons such as benzene, toluene or benzotrifluoride; ethers such as diethyl ether, diisopropyl ether, tetrahydrofuran, dioxane, dimethoxyethane or diethylene glycol dimethyl ether; halogenated hydrocarbons or aromatic hydrocarbons are preferable, dichloromethane, chloroform, or toluene being most preferable. Examples of the chlorinating agent used include methanesulfonyl chloride, oxalic acid chloride, thionyl chloride, phosphorus oxychloride, phosphorus trichloride, phosphorus pentachloride, and hydrochloric acid; thionyl chloride or hydrochloric acid is preferable. Examples of the base used include tertiary alkyl amines such as trimethylamine and triethylamine, and pyridine; triethylamine being preferable.

The reaction temperature depends on the starting material, the solvent, and the chlorinating agent, but is generally in a range of from −20 to 30° C., preferably from 0 to 10° C.

The reaction time depends on the starting material, the solvent, the chlorinating agent, and the reaction temperature, but is generally in a range of from 10 minutes to 6 hours, preferably from 10 minutes to 2 hours.

The compound obtained in the present step can be used as is in the next step without being isolated in particular.

In the case of brominating, a reagent such as bromine/red phosphorus, phosphorus tribromide or phosphorus pentabromide can be used, or in the case of iodinating, a reagent such as iodine/red phosphorus can be used. Alternatively, a brominated compound or iodinated compound can be obtained by making a reagent such as sodium bromide or sodium iodide act on the leaving group in the compound synthesized in step P1.

### Step P2: Thioetherification

The present step is a step of reacting compound (5T) with compound (6T) or a salt thereof (particularly a hydrochloride) either in the presence of or without a base and either without a solvent or in an inert solvent, so as to produce compound (3T).

There are no particular limitations on a solvent used so long as this is a solvent in which the starting materials dissolve to some extent and that does not impede the reaction. Examples of the solvent include alcohols such as methanol, ethanol, n-propanol, isopropanol, n-butanol, isobutanol, t-butanol, isoamyl alcohol, diethylene glycol, glycerol, octanol, cyclohexanol or methyl cellosolve; halogenated hydrocarbons such as chloroform, dichloromethane, 1,2-dichloroethane or carbon tetrachloride; ethers such as diethyl ether, diisopropyl ether, tetrahydrofuran, dioxane, dimethoxyethane or diethylene glycol dimethyl ether; aromatic hydrocarbons such as benzene or toluene; N,N-dimethylformamide; dimethyl sulfoxide; water; or a mixed solvent thereof; dichloromethane, alcohols, ethers, or a mixed solvent of an ether and toluene are preferable, methanol, tetrahydrofuran, or a mixed solvent of tetrahydrofuran and toluene being most preferable.

Examples of the base used include inorganic bases such as sodium hydride, potassium hydride, lithium carbonate, sodium carbonate, potassium carbonate, lithium hydroxide, sodium hydroxide and potassium hydroxide; and organic bases such as N-methylmorpholine, triethylamine, tripropylamine, tributylamine, diisopropylethylamine, dicyclohexylamine, N-methylpiperidine, pyridine, 4-pyrrolidinopyridine, picoline, 4-(N,N-dimethylamino)pyridine, 2,6-di(t-butyl)-4-methylpyridine, quinoline, N,N-dimethylaniline, N,N-diethylaniline, 1,5-diazabicyclo[4.3.0]non-5-ene (DBN), 1,4-diazabicyclo[2.2.2]octane (DABCO) and 1,8-diazabicyclo[5.4.0]undec-7-ene (DBU); inorganic bases such as sodium hydride, potassium hydride, lithium hydroxide, sodium hydroxide or potassium hydroxide, or triethylamine is preferable, sodium hydroxide or triethylamine being most preferable.

The reaction temperature depends on the starting materials, the solvent, and the base, but is generally in a range of from 0 to 100° C., preferably from 10 to 50° C.

The reaction time depends on the starting materials, the solvent, the base, and the reaction temperature, but is generally in a range of from 30 minutes to 3 days.

### Step P5: Ether Group Introduction Reaction

The present step is a step of reacting compound (3T) with the alcohol represented by the following formula:

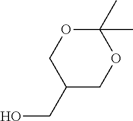

((2,2-dimethyl-1,3-dioxan-5-yl)methanol or a hydrate thereof etc.) in the presence of a base either without a solvent or in an inert solvent, so as to produce compound (2T).

There are no particular limitations on a solvent used so long as this is a solvent in which the starting materials dissolve to some extent and that does not impede the reaction. Examples of the solvent include aliphatic hydrocarbons such as hexane, heptane, ligroin or petroleum ether; halogenated hydrocarbons such as chloroform, dichloromethane, 1,2-dichloroethane or carbon tetrachloride; aromatic hydrocarbons such as benzene or toluene; ethers such as diethyl ether, diisopropyl ether, tetrahydrofuran, dioxane, dimethoxyethane or diethylene glycol dimethyl ether; amides such as formamide, N,N-dimethylformamide, N,N-dimethylacetamide, hexamethylphosphoric acid triamide or N-methylpyrrolidone, dimethyl sulfoxide, or water, or a mixed solvent thereof; dimethyl sulfoxide, an ether, or an amide is preferable, dimethyl sulfoxide being most preferable.

Examples of the base used include alkali metal carbonates such as lithium carbonate, sodium carbonate and potassium carbonate; alkali metal hydroxides such as lithium hydroxide, sodium hydroxide and potassium hydroxide; metal alkoxides such as lithium methoxide, sodium methoxide, sodium ethoxide and potassium t-butoxide; alkali metal hydrides such as lithium hydride, sodium hydride and potassium hydride; alkali metal alkoxides prepared using an alkali metal, n-butyllithium, and lithium diisopropylamide; metal alkoxides or alkali metal hydrides are preferable, potassium t-butoxide or sodium hydride being most preferable.

The reaction temperature depends on the starting materials, the solvent, and the base, but is generally in a range of from 0 to 160° C., preferably from 20 to 140° C.

The reaction time depends on the starting materials, the solvent, the base, and the reaction temperature, but is generally in a range of from 15 minutes to 96 hours, preferably from 30 minutes to 72 hours.

### Step P6: Oxidation Reaction

The present step is a step of reacting compound (2T) with an oxidizing agent either without or in the presence of a solvent, so as to produce compound (1).

There are no particular limitations on a solvent used so long as this is a solvent in which the starting material dissolves to some extent and that does not impede the reaction. Examples of the solvent include alcohols such as methanol, ethanol, n-propanol, isopropanol, n-butanol, isobutanol, t-butanol, isoamyl alcohol, diethylene glycol, glycerol, octanol, cyclohexanol or methyl cellosolve; aromatic hydrocarbons such as benzene or toluene; halogenated hydrocarbons such as chloroform, dichloromethane, 1,2-dichloroethane or carbon tetrachloride; amides such as formamide, N,N-dimethylformamide, N,N-dimethylacetamide or hexamethylphosphoric acid triamide; or nitriles such as acetonitrile; aromatic hydrocarbons, alcohols, or halogenated hydrocarbons, or a mixed solvent thereof is preferable, toluene, a mixed solvent of toluene and methanol, or dichloromethane being most preferable.

Examples of the oxidizing agent used include hydrogen peroxide, t-butyl hydroperoxide, cumene hydroperoxide, sodium periodate, peracetic acid, perbenzoic acid, 3-chloroperbenzoic acid, and a urea hydrogen peroxide adduct ((NH₂)₂CO.H₂O₂); 3-chloroperbenzoic acid or cumene hydroperoxide is preferable.

Note that asymmetric oxidation may be carried out using a method described, for example, in any of the following documents: WO 96/02535, WO 2001/83473, WO 2004/087702, WO 2004/052881, WO 2004/052882, Adv. Synth. Catal. 2005, 347, 19-31, Chem. Rev. 2003, 103, 3651-3705, Tetrahedron Lett. 2004, 45, 9249-9252, Angew. Chem. Int. Ed. 2004, 43, 4225-4228, or Tetrahedron Asymmetry 2003, 14, 407-410.

More specifically, asymmetric oxidation is carried out by reacting the compound (2T) and the oxidizing agent together in the presence of an asymmetry induction agent or an asymmetry induction catalyst.

Examples of the oxidizing agent include peroxides such as hydrogen peroxide, tert-butyl hydroperoxide, urea hydroperoxide or cumene hydroperoxide; in particular cumene hydroperoxide is used in the case that the asymmetry induction agent or asymmetry induction catalyst contains titanium, zirconium or hafnium, whereas a hydrogen peroxide aqueous solution is used in the case that the asymmetry induction agent or asymmetry induction catalyst contains vanadium.

The amount used of the oxidizing agent is preferably an excess with respect to compound (2T), preferably from 1.01 to 10 molar equivalents. In particular, 1.05 molar equivalents is generally used in the case that the asymmetry induction agent or asymmetry induction catalyst contains titanium, 1.2 molar equivalents in the case that the asymmetry induction agent or asymmetry induction catalyst contains zirconium or hafnium, and 1.1 molar equivalents in the case that the asymmetry induction agent or asymmetry induction catalyst contains vanadium.

Examples of the asymmetry induction agent or asymmetry induction catalyst include:

(1) optically active titanium complexes such as a complex between an optically active diol, a titanium (IV) alkoxide, and water or an alcohol;

(2) optically active zirconium complexes such as a complex between an optically active diol and a zirconium (IV) alkoxide (water being optionally contained);

(3) optically active hafnium complexes such as a complex between an optically active diol and a hafnium (IV) alkoxide;

(4) optically active vanadium complexes such as a complex between an optically active Schiff base and vanadyl acetylacetone;

(5) optically active iron complexes such as a complex between an optically active Schiff base and iron (III) acetylacetonate;

(6) optically active manganese complexes of manganese (III) with an optically active Schiff base (e.g. a salen-manganese complex); and

(7) optically active tungsten complexes of tungsten (III) with an optically active cinchona alkaloid.

Examples of the above optically active diol include:

(1) alkyl diols such as tartaric acid esters such as (+) or (−) dimethyl tartrate, diethyl tartrate, diisopropyl tartrate and dibutyl tartrate, and tartaric acid amides such as tartaric acid tetramethyldiamide; and
(2) aromatic diols such as (R)- or (S)-binaphthol.

Examples of the above optically active Schiff base include Schiff bases derived from substituted salicyl aldehyde such as (S)-(−)-2-(3,5-di-tert-butylsalicylideneamino)-3,3-dimethyl-1-butanol and (1R,2S)-1-[(2-hydroxy-3,5-di-tert-butylbenzylidene)amino]indan-2-ol, and salen type Schiff bases.

When carrying out the asymmetric oxidation, a base may be added as required. There are no particular limitations on the base used so long as the base does not impede the reaction. Examples of the base include inorganic bases and organic bases; tertiary amines such as diisopropylethylamine or triethylamine are preferable, diisopropylethylamine being most preferable. The amount of the base is generally from 0.1 to 1 equivalents with respect to compound (2T).

Note that in the case of using the asymmetry induction agent or asymmetry induction catalyst containing vanadium, the base is generally not used.

Examples of the solvent used when carrying out the asymmetric oxidation include aromatic hydrocarbons such as toluene, benzene or xylene; halogenated hydrocarbons such as dichloromethane or chloroform; or esters such as ethyl acetate; in particular, in the case of using the asymmetry induction agent or asymmetry induction catalyst containing titanium, zirconium or hafnium, toluene or tert-butyl methyl ether is preferable, whereas in the case of using the asymmetry induction agent or asymmetry induction catalyst containing vanadium, acetonitrile or dichloromethane is preferable. Moreover, in the case of using the asymmetry induction catalyst containing titanium, it is effective to add water, the amount of water added preferably being from 0.1 to 0.33 equivalents, most preferably from 0.13 to 0.25 equivalents, with respect to compound (2T) including the water content of the solvent, the reactants (excluding the oxidizing agent) and the substrate. Moreover, the amount of water can be controlled by using a 3 A molecular sieve.

When synthesizing a complex between a titanium (IV) alkoxide and an alcohol, isopropanol is effective as the alcohol used, the isopropanol generally being used in an amount of 1.2 equivalents with respect to titanium.

The reaction temperature depends on the starting material, the solvent, and the oxidizing agent, but is generally in a range of from −100 to 100° C., preferably from −70 to 70° C.

The reaction time depends on the starting material, the solvent, the oxidizing agent, and the reaction temperature, but is generally in a range of from 15 minutes to 72 hours, preferably from 30 minutes to 24 hours.

Moreover, the compound obtained as described above can be made into a salt using the conventional method. An example is reacting the compound (1) with a base either without or in the presence of a solvent. Examples of the solvent include acetonitrile; alcohols such as methanol or ethanol; water; or a mixed solvent thereof; a mixed solvent of ethanol and water is preferable. Examples of the base include alkali metal hydroxides such as lithium hydroxide, sodium hydroxide or potassium hydroxide; alkaline earth metal hydroxides such as magnesium hydroxide; alkoxides such as sodium methoxide, sodium t-butoxide, sodium t-pentoxide or magnesium methoxide, it being preferable to use sodium hydroxide in the form of an aqueous solution. The reaction temperature is generally in a range of from −50 to 50° C., preferably from 10 to 40° C. The reaction time is generally in a range of from 1 minute to 2 hours, preferably from 1 minute to 1 hour.

Moreover, alkali metal salts such as a sodium salt or a potassium salt may be subjected to a salt exchange reaction with metal chlorides or metal sulfates such as barium chloride, magnesium chloride, magnesium sulfate or zinc sulfate, either without or in the presence of solvent, and thus converted into the corresponding metal salt such as a barium salt, a magnesium salt or a zinc salt.

Moreover, after the oxidation of compound (2T), the compound (1) may be subjected to a salt-forming reaction without first being isolated, so as to obtain a metal salt.

### Step P3: Oxidation Reaction

The present step is a step of reacting compound (3T) with an oxidizing agent either without or in the presence of a solvent, so as to produce compound (4T). As the reaction conditions for the present step, conditions and a procedure as for step P6 described above can be used.

### Step P4: Ether Group Introduction Reaction

The present step is a step of reacting compound (4T) with the alcohol ((2,2-dimethyl-1,3-dioxan-5-yl)methanol or a hydrate thereof etc.) in the presence of a base either without a solvent or in an inert solvent, so as to produce compound (1). As the reaction conditions for the present step, conditions and a procedure as for step P5 described above can be used.

As compound (5T) and compound (7T), which are intermediates in the method P described above, commercially available compounds may be used, or else compound (5T) and compound (7T) can be easily produced from commercially available compounds using the process ordinarily carried out by persons skilled in the art. In particular, compound (7T) can be produced using the method V described later.

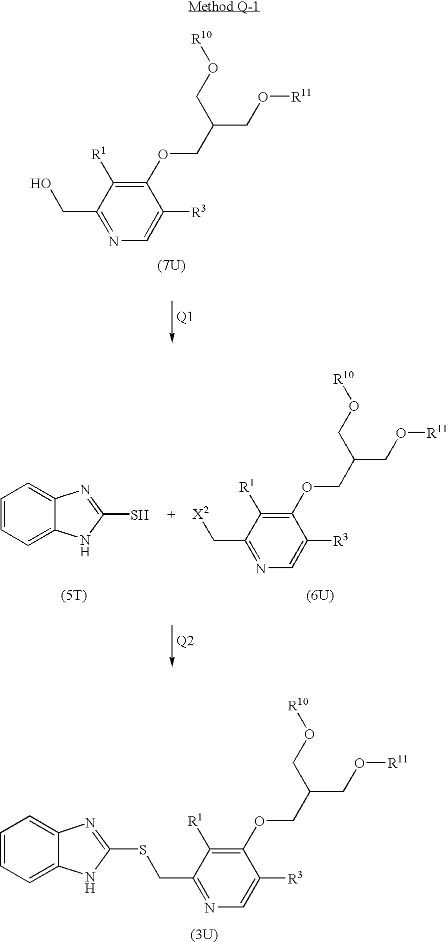

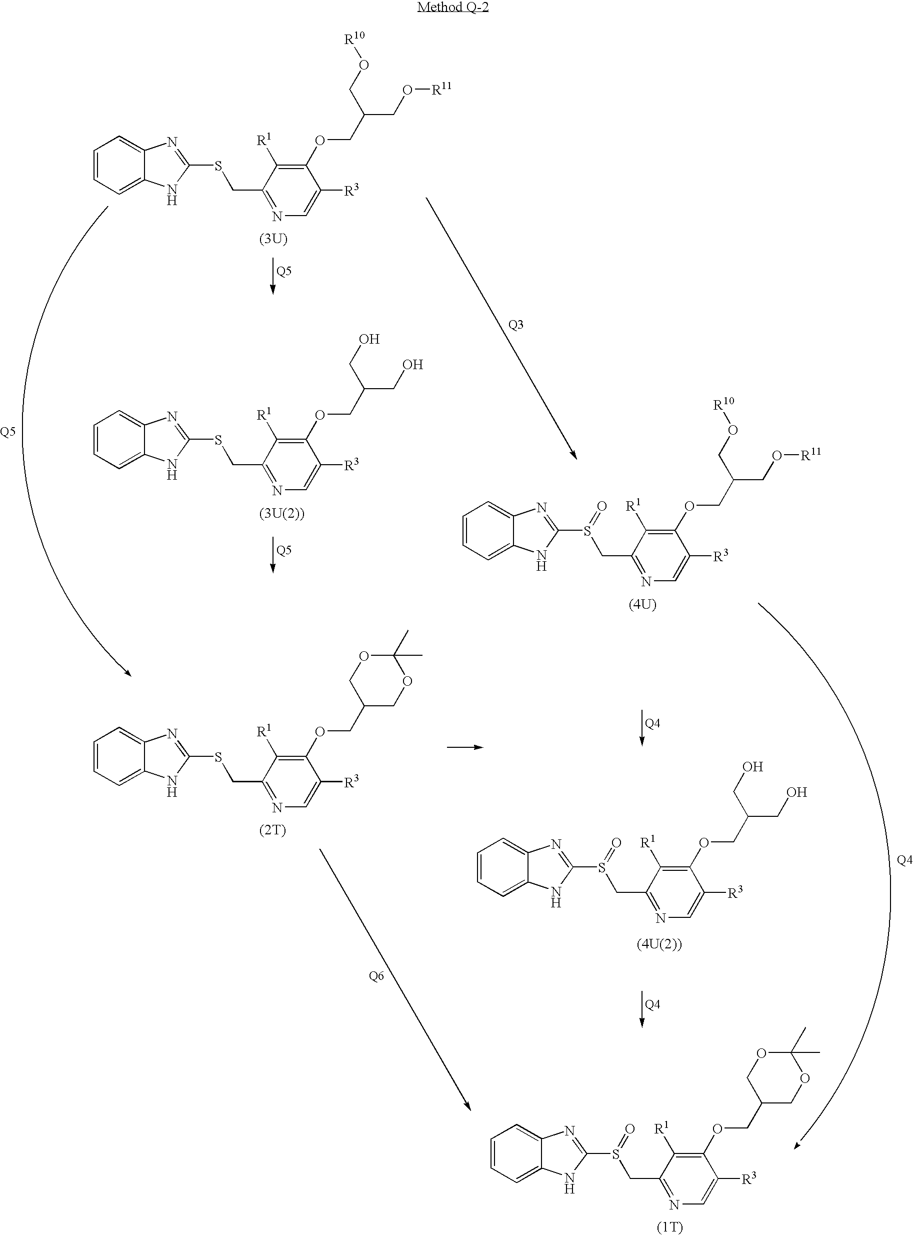

In the above, R1, R3, R10 and R11 are defined as above, and X2 represents a leaving group.

Examples of the leaving group X2 are sulfonyloxy groups such as methanesulfonyloxy, p-toluenesulfonyloxy and trifluoromethanesulfonyloxy; halogen groups such as chlorine, bromine and iodine; and acyloxy groups such as acetyloxy, trifluoroacetyloxy and propionyloxy; methanesulfonyloxy, p-toluenesulfonyloxy, chlorine, or an acetyloxy group is preferable.

Following is a description of each of the steps in the method Q.

### Step Q1: Introduction of Leaving Group or Halogenation

The present step is a step of reacting compound (7U) and a leaving group introducing agent (e.g. a chlorinating agent) together in the presence of a base either without a solvent or in an inert solvent, so as to produce compound (6U) or a salt thereof. As the reaction conditions for the present step, conditions and procedures as for step P1 described above can be used.

### Step Q2: Thioetherification

The present step is a step of reacting compound (5T) with compound (6U) or a salt thereof (particularly a hydrochloride) either in the presence of or without a base and either without a solvent or in an inert solvent, so as to produce compound (3U). As the reaction conditions for the present step, conditions and procedures as for step P2 described above can be used.

### Step Q3: Oxidation Reaction

The present step is a step of reacting compound (3U) with an oxidizing agent either without or in the presence of a solvent, so as to produce compound (4U). As the reaction conditions for the present step, conditions and procedures as for step P6 described above can be used.

### Step Q5: Acetal-Forming Reaction

The present step is a step of eliminating R10 and R11 in compound (3U), and then acetalizing so as to produce compound (2T), this being either without or in the presence of a solvent, or a step of simultaneously eliminating R10 and R11 in compound (3U) and acetalizing so as to produce compound (2T).

R10 and R11 in compound (3U) may be eliminated so as to produce compound (3U(2)) (protecting group deprotection step), before then producing compound (2T) from compound (3U(2)) (acetalization step), or alternatively compound (2T) may be produced by eliminating R10 and R11 in compound (3U) and carrying out the acetalization reaction substantially simultaneously with this.

### Step Q5 (1): Protecting Group Deprotection Step

This is a step of eliminating R10 and R11 in compound (3U) so as to obtain compound (3U(2)).

This protecting group deprotection step can be carried out under general deprotection reaction conditions suitable for R10 and R11.

For example, in the case that R10 and R11 are combined so as to represent a methylene group (the methylene group optionally having thereon one or two groups selected from C1-C6 alkyl groups, C1-C6 alkoxy groups, phenyl groups optionally having thereon one or two methoxy groups, and trichloromethyl groups), a 1,1-cyclopropylene group, a 1,1-cyclobutylene group, a 1,1-cyclopentylene group, a 1,1-cyclohexylene group, or a tetrahydropyranyl group, compound (3U(2)) can be obtained from compound (3U) by reacting in the presence of an acid.

There are no particular limitations on a solvent used so long as this is a solvent in which the starting material dissolves to some extent and that does not impede the reaction. Examples of the solvent include methanol, tetrahydrofuran, a mixed solvent thereof or the like, methanol being preferable.

Examples of the acid used include hydrochloric acid, trifluoroacetic acid, p-toluenesulfonic acid or the like, hydrochloric acid being preferable.

The reaction temperature depends on the starting material and the solvent, but is generally in a range of from 0 to 100° C., preferably from 10 to 50° C.

The reaction time depends on the starting material, the solvent, and the reaction temperature, but is generally in a range of from 30 minutes to 3 days.

In the case that each of R10 and R11 represents a benzyl group or the like optionally substituted with a methoxy group, a nitro group or the like, compound (3U(2)) obtained through the deprotection of R10 and R11 can be obtained by reacting with a reducing reagent.

In the case that each of R10 and R11 represents a C1-C6 acyl group (an acyl group etc.), compound (3U(2)) obtained through the deprotection of R10 and R11 can be obtained by reacting in the presence of a base.

In the case that each of R10 and R11 represents a trimethylsilyl group, a t-butyldimethylsilyl group or the like, compound (3U(2)) obtained through the deprotection of R10 and R11 can be obtained by reacting in the presence of a fluorine reagent (e.g. tetrabutylammonium fluoride).

In this way, to eliminate (deprotect) R10 and R11 in compound (3U), there can be used reaction conditions for elimination (reaction conditions for eliminating protecting groups) generally used in organic synthesis, this being in accordance with R10 and R11 as appropriate (Protective Groups in Organic Synthesis Third Edition, John Wiley & Sons, Inc. 1999).

The compound obtained in the present step can be used as is in the next step without being isolated in particular.

### Step Q5 (2): Acetalization Step

The present step is a step of reacting compound (3U(2)) with an acetalizing reagent either in the presence of or without an acid and either without or in the presence of a solvent, so as to produce compound (2T).

This acetalization step can be carried out under general acetalization reaction conditions (in particular diol compound acetal protection reaction conditions).

There are no particular limitations on a solvent used so long as this is a solvent in which the starting material dissolves to some extent and that does not impede the reaction. Examples of the solvent include amides such as formamide, N,N-dimethylformamide, N,N-dimethylacetamide or hexamethylphosphoric acid triamide, halogenated hydrocarbons such as chloroform, dichloromethane, 1,2-dichloroethane or carbon tetrachloride, ethers such as diethyl ether, diisopropyl ether, tetrahydrofuran, dioxane, dimethoxyethane or diethylene glycol dimethyl ether, or a mixed solvent thereof; dichloromethane or N,N-dimethylformamide is preferable, N,N-dimethylformamide being most preferable.

Examples of the acid used include p-toluenesulfonic acid, hydrochloric acid, sulfuric acid and iron (III) chloride; p-toluenesulfonic acid, hydrochloric acid, sulfuric acid or iron (III) chloride being preferable, iron (III) chloride being most preferable.

The acetalizing reagent used means a reagent for forming an isopropylidene ketal (—CH(CH₃)₂—). Specific examples of the acetalizing reagent include (1) acetone, (2) compounds represented by the formula (CH₃)₂C(OR^{1X})₂ (wherein R^{1X} represents a C1-C6 alkyl group or a trimethylsilyl group), and (3) compounds represented by the formula (CH₃)C(OR^{1X})═CH₂ (wherein R^{1X} represents a C1-C6 alkyl group or a trimethylsilyl group); acetone, 2,2-diethoxypropane, or 2-methoxypropene is preferable.

The reaction temperature depends on the starting material and the solvent, but is generally in a range of from 0 to 150° C., preferably from 10 to 120° C.

The reaction time depends on the starting material, the solvent, and the reaction temperature, but is generally in a range of from 30 minutes to 3 days.

The compound obtained in the present step can be used as is in the next step without being isolated in particular.

Referring to the conditions for the protecting group deprotection step and the conditions for the acetalization step described above, the acetalization reaction may alternatively be carried out substantially simultaneously with the elimination of R10 and R11 in compound (3U), so as to produce compound (2T).

### Step Q4: Acetal-Forming Reaction

The present step is a step of eliminating R10 and R11 in compound (4U), and then acetalizing so as to produce compound (1T), this being either without or in the presence of a solvent, or a step of simultaneously eliminating R10 and R11 in compound (4U) and acetalizing so as to produce compound (1T). As the reaction conditions for the present step, conditions and procedures as for step Q5 described above can be used.

### Step Q6: Oxidation Reaction

The present step is a step of reacting compound (2T) with an oxidizing agent either without or in the presence of a solvent, so as to produce compound (1T). As the reaction conditions for the present step, conditions and procedures as for step P6 described above can be used.

As compound (5T), which is an intermediate in the method Q described above, a commercially available compound may be used, or compound (5T) can be easily produced from a commercially available compound using a process ordinarily carried out by persons skilled in the art. Moreover, compound (7U) may be produced using the method V described below.

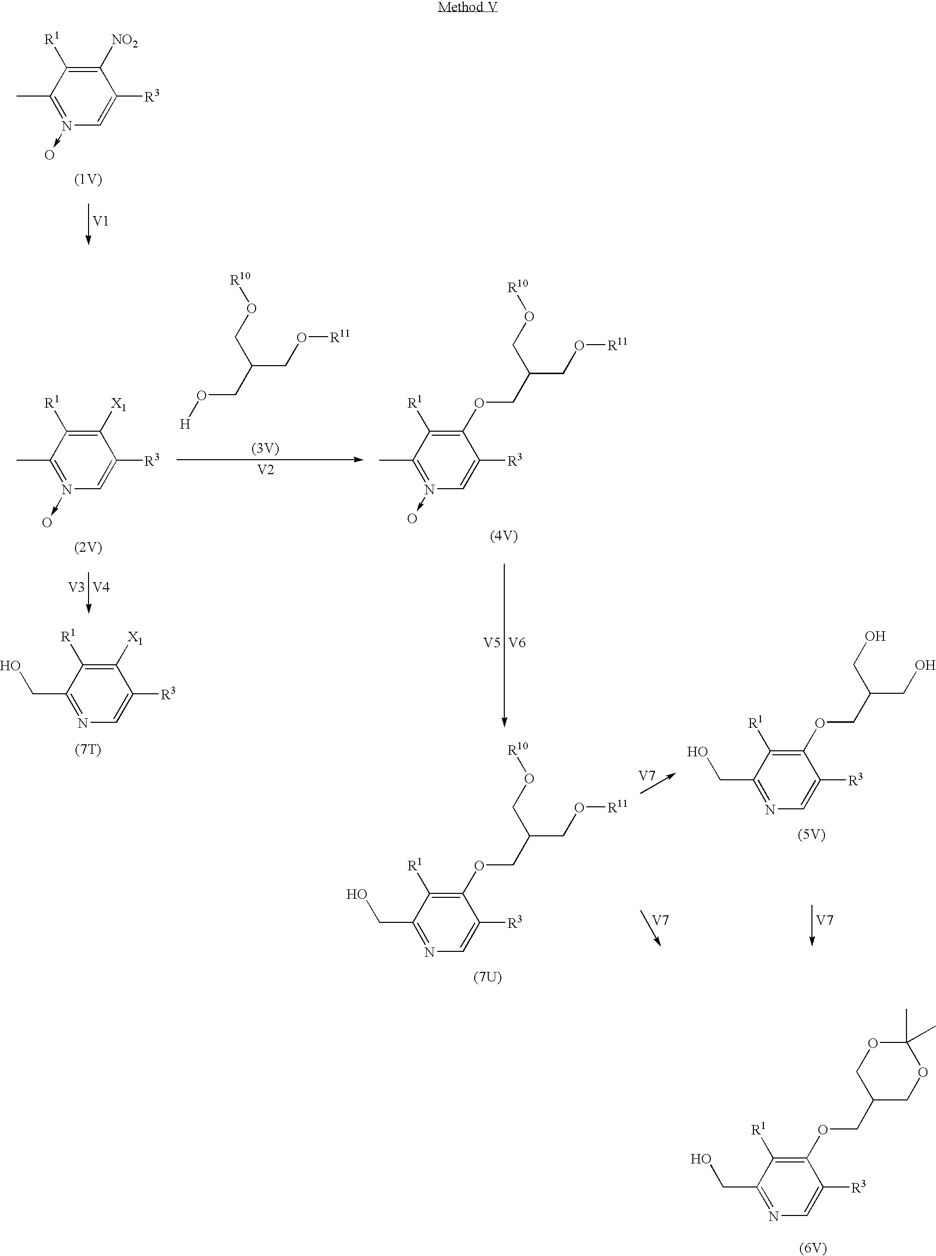

In the above, R1, R3, R10 and R11 are defined as above, and X1 represents a leaving group. Specific examples of the leaving group include sulfonyloxy groups such as methanesulfonyloxy, p-toluenesulfonyloxy and trifluoromethanesulfonyloxy, halogen groups such as chlorine, bromine and iodine, acyloxy groups such as acetyloxy, trifluoroacetyloxy and propionyloxy, sulfonyl groups such as benzenesulfonyl, and a nitro group; a chlorine atom, a bromine atom, an iodine atom; a nitro group is preferable, a chlorine atom being particularly preferable.

Following is a description of each of the steps in the method V.

### Step V1: Halogenation Reaction (Taking a Chlorination Reaction as a Representative Example)

The present step is a step of reacting compound (1V) with a chlorinating agent either without a solvent or in an inert solvent, so as to produce compound (2V).

In the present step, it is generally preferable to carry out the reaction in the chlorinating agent without using a solvent, but in the case that a solvent is used, there are no particular limitations on the solvent used so long as this is a solvent in which the starting material dissolves to some extent and that does not impede the reaction. Examples of the solvent include halogenated hydrocarbons such as chloroform, dichloromethane, 1,2-dichloroethane or carbon tetrachloride; ethers such as diethyl ether, diisopropyl ether, tetrahydrofuran, dioxane, dimethoxyethane or diethylene glycol dimethyl ether.

Examples of the chlorinating agent used include acetyl chloride, oxalic acid chloride, thionyl chloride, phosphorus oxychloride, phosphorus trichloride, and phosphorus pentachloride; acetyl chloride is preferable.

The reaction temperature depends on the starting material, the solvent, and the chlorinating agent, but is generally in a range of from −50 to 30° C., preferably from −30 to 10° C.

The reaction time depends on the starting material, the solvent, the chlorinating agent, and the reaction temperature, but is generally in a range of from 30 minutes to 8 hours, preferably from 1 to 5 hours.

In the case of brominating, a reagent such as acetyl bromide, hydrogen bromide, bromine/red phosphorus, phosphorus tribromide or phosphorus pentabromide can be used, or in the case of iodinating, a reagent such as iodine/red phosphorus can be used, or alternatively bromination may be carried out followed by reaction with a reagent such as sodium iodide.

### Step V2: Ether Group Introduction Reaction

The present step is a step of reacting compound (2V) with an alcohol represented by formula (3V) in the presence of a base either without a solvent or in an inert solvent, so as to produce compound (4V).

There are no particular limitations on a solvent used so long as this is a solvent in which the starting material dissolves to some extent and that does not impede the reaction. Examples of the solvent include aliphatic hydrocarbons such as hexane, heptane, ligroin or petroleum ether; halogenated hydrocarbons such as chloroform, dichloromethane, 1,2-dichloroethane or carbon tetrachloride, aromatic hydrocarbons such as benzene or toluene; ethers such as diethyl ether, diisopropyl ether, tetrahydrofuran, dioxane, dimethoxyethane or diethylene glycol dimethyl ether; amides such as formamide, N,N-dimethylformamide, N,N-dimethylacetamide, hexamethylphosphoric acid triamide or N-methylpyrrolidone; dimethyl sulfoxide, or water, or a mixed solvent thereof; dimethyl sulfoxide, an ether, or an amide is preferable, dimethyl sulfoxide being most preferable.

Examples of the base used include alkali metal carbonates such as lithium carbonate, sodium carbonate and potassium carbonate; alkali metal hydroxides such as lithium hydroxide, sodium hydroxide and potassium hydroxide; metal alkoxides such as lithium methoxide, sodium methoxide, sodium ethoxide and potassium t-butoxide; alkali metal hydrides such as lithium hydride, sodium hydride and potassium hydride; alkali metal alkoxides prepared using alkali metals, n-butyllithium, and lithium diisopropylamide; alkali metal hydrides are preferable, sodium hydride being most preferable.

The reaction temperature depends on the starting material, the solvent, and the base, but is generally in a range of from 0 to 100° C., preferably from 10 to 100° C.

The reaction time depends on the starting material, the solvent, the base, and the reaction temperature, but is generally in a range of from 15 minutes to 48 hours, preferably from 30 minutes to 12 hours.

Step V3: Rearrangement into Acetic Acid Ester

The present step is a step of reacting compound (2V) with acetic anhydride either in the presence of or without a base and without a solvent, so as to produce an acetic acid ester of compound (7T).

Examples of a base used include tertiary alkyl amines such as trimethylamine, diisopropylethylamine and triethylamine, and pyridine; triethylamine is preferable.

The reaction temperature depends on the starting material and the solvent, but is generally in a range of from 20 to 150° C., preferably from 20 to 60° C. in the presence of a base, or from 50 to 100° C. without a base.

The reaction time depends on the starting material, the solvent, and the reaction temperature, but is generally in a range of from 10 minutes to 6 hours, preferably from 30 minutes to 5 hours.

After the reaction, residue obtained by evaporating off the acetic anhydride can generally be used as is in the next step. Alternatively, compound (3T) may be obtained from the acetic acid ester by carrying out step P2 of the method P described earlier.

### Step V4: Hydrolysis Reaction

The present step is a step of reacting the compound obtained in above step V4 with a base either without or in the presence of a solvent, so as to produce compound (7T).

There are no particular limitations on a solvent used so long as this is a solvent in which the starting material dissolves to some extent and that does not impede the reaction. Examples of the solvent include water, alcohols such as methanol, ethanol, n-propanol, isopropanol, n-butanol, isobutanol, t-butanol, isoamyl alcohol, diethylene glycol, glycerol, octanol, cyclohexanol or methyl cellosolve; aliphatic hydrocarbons such as hexane, heptane, ligroin or petroleum ether; ethers such as diethyl ether, diisopropyl ether, tetrahydrofuran, dioxane, dimethoxyethane or diethylene glycol dimethyl ether; halogenated hydrocarbons such as chloroform, dichloromethane, 1,2-dichloroethane or carbon tetrachloride, or amides such as formamide, N,N-dimethylformamide, N,N-dimethylacetamide or hexamethylphosphoric acid triamide, or a mixed solvent thereof; alcohol, or a mixed solvent of an alcohol and water is preferable, a mixed solvent of methanol and water being most preferable.

Examples of the base used include alkali metal carbonates such as lithium carbonate, sodium carbonate and potassium carbonate; alkali metal hydroxides such as lithium hydroxide, sodium hydroxide and potassium hydroxide; metal alkoxides such as lithium methoxide, sodium methoxide, sodium ethoxide and potassium t-butoxide, and ammonia preparations such as ammonia water or concentrated ammonia-methanol; alkali metal hydroxides are preferable, sodium hydroxide being most preferable.

The reaction temperature depends on the starting material, the solvent, and the base, but is generally in a range of from 0 to 60° C., preferably from 10 to 40° C.

The reaction time depends on the starting material, the solvent, the base, and the reaction temperature, but is generally in a range of from 10 minutes to 6 hours.

Step V5: Rearrangement into Acetic Acid Ester

The present step is a step of reacting compound (4V) with acetic anhydride either in the presence of or without a base and without a solvent, so as to produce an acetic acid ester of compound (7U). As the reaction conditions for the present step, conditions and procedures as for step V3 described above can be used.

### Step V6: Hydrolysis Reaction

The present step is a step of reacting the compound obtained in above step V5 with a base either without or in the presence of a solvent, so as to produce compound (7U). As the reaction conditions for the present step, conditions and procedures as for step V4 described above can be used.

### Step V7: Acetal-Forming Reaction

The present step is a step of eliminating R10 and R11 in compound (7U), and then acetalizing so as to produce compound (6V), this being either without or in the presence of a solvent, or a step of simultaneously eliminating R10 and R11 in compound (7U) and acetalizing so as to produce compound (6V). As the reaction conditions for the present step, conditions and procedures as for step Q5 described above can be used.

In each of the steps in each of the methods described above, after completion of the reaction, the target compound for that step can be recovered from the reaction mixture using an ordinary method.

In the case of using compound (1) or a salt thereof as a drug, the compound (1) or salt thereof is generally used in the form of a preparation with suitable additives mixed therewith. Note, however, that the case of the compound (1) or salt thereof being used as a drug as is in its original form is not excluded.

Examples of additives include excipients, binders, lubricants, disintegrators, colorants, flavor/odor improvers, emulsifiers, surfactants, solubilizers, suspending agents, isotonizing agents, buffering agents, preservatives, antioxidants, stabilizers and absorption enhancers generally used in drugs; an appropriate combination of these may be used as desired.

### EXAMPLES

The present invention will be described in more detail below, giving Examples. However, the following description is merely illustrative, and the present invention is not limited thereto in any case. In the chemical formulae in the Examples, atoms marked * represents asymmetric atoms.

### Cyclobutanone (Avocado)

2-(hydroxymethyl)-1,3-propanediol (Aldrich)
p-toluenesulfonic acid monohydrate (Tokyo Chemical Industry)
Potassium hydroxide (Wako Pure Chemical Industries)
Acetic anhydride (Kanto Chemical)
5N sodium hydroxide aqueous solution (Wako Pure Chemical Industries)
Methanesulfonyl chloride (Tokyo Chemical Industry)

### Triethylamine (Kanto Chemical)

2-mercaptobenzimidazole (Tokyo Chemical Industry)
Sodium hydroxide (Wako Pure Chemical Industries)
2N hydrochloric acid (Wako Pure Chemical Industries)
2,2-diethoxypropane (Tokyo Chemical Industry)
Iron (III) chloride (Wako Pure Chemical Industries)

### Acetone (Wako Pure Chemical Industries)

3-Chloroperbenzoic acid (Tokyo Chemical Industry)
1N sodium hydroxide aqueous solution (Wako Pure Chemical Industries)

### Production Examples

### (1) (2,2-Dimethyl-1,3-dioxan-5-yl)methanol

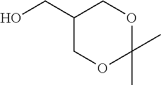

A mixture of 2-(hydroxymethyl)-1,3-propanediol (4.09 g, 38.5 mmol), acetone (130 ml, 1768 mmol), and 70% perchloric acid (1.37 g, 9.55 mmol) was stirred at room temperature for 21 hours. A pH of the reaction mixture was adjusted to 9 using concentrated ammonia water, and then subjected to concentration. The residue was purified by silica gel column chromatography (silica gel: 100 g, eluent: heptane, heptane/ethyl acetate=1/3), whereby the title compound (4.83 g, percentage yield 85.8%) was obtained as a colorless oil.

¹H-NMR (400 MHz, DMSO-d6) δ ppm: 1.29 (3H, s), 1.30 (3H, s), 1.64-1.74 (1H, m), 3.35-3.41 (2H, m), 3.61 (2H, dd, J=7, 12 Hz), 3.82 (2H, dd, J=4, 12 Hz), 4.54 (1H, t, J=5 Hz).

### (2) 2,3,5-Trimethylpyridine 1-oxide

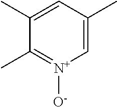

2,3,5-trimethylpyridine (1.43 kg, 11.80 mol) was added to acetic acid (1.43 kg, 23.83 mol) over 15 minutes. After 15 minutes, a 35% hydrogen peroxide aqueous solution (1.38 kg, 14.2 mol) was added dropwise in over 30 minutes, followed by stirring at from 90 to 95° C. overnight. Sodium sulfite (220 g) was then added to the reaction liquid. The reaction mixture was then added to a mixture of sodium carbonate (2.5 kg) and water (12 L), and extraction was carried out with chloroform (3.0 L×4). The resulting organic layers were subjected to concentration until crystals precipitated out, and then n-hexane (2.5 L) was added to the precipitate, and stirring was carried out in a cooling-ice overnight. The resulting crystals were filtered off, whereby 1.53 kg of the target substance was obtained.

### (3) 4-Nitro-2,3,5-trimethylpyridine 1-oxide

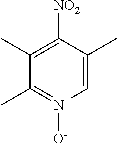

2,3,5-Trimethylpyridine 1-oxide (1.38 kg, 10.1 mol) was added to 98% sulfuric acid (4.93 kg, 49.3 mol). 97% Nitric acid (1.44 kg) was added dropwise over 50 minutes, and then heating was carried out at 85° C. for 4 hours. The reaction liquid was then added to a mixture of ammonium hydrogencarbonate (10.6 kg) and water (9.0 L), and extraction was carried out with ethyl acetate (3.0 L×3). The resulting organic layers were subjected to concentration, and vacuum drying was carried out overnight, whereby 1.50 kg of the target substance was obtained.

### (4) 4-Chloro-2,3,5-trimethylpyridine 1-oxide

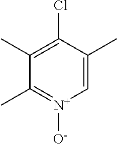

To 4-nitro-2,3,5-trimethylpyridine 1-oxide (850 g, 4.67 mol) were added water (400 g) and 36% hydrochloric acid (1.69 kg), which was heated to 70° C. N,N-dimethylformamide (115 mL) was added thereto, followed by heating to 100° C. After completion of the reaction, the reaction mixture was cooled to 20° C., and was added to a mixture of potassium carbonate (1.40 kg) and water (7 L), and then extraction was carried out with chloroform (1.0 L×3), and then after drying with sodium sulfate, concentration was carried out. The resulting crude material was stirred for 2 hours in a mixture of diisopropyl ether (500 mL) and n-hexene (1.0 L), and then suction filtration was carried out. The resulting wet material was subjected to vacuum drying overnight, whereby 666.4 g of the target substance was obtained.

### (5) 4-(2,2-Dimethyl-1,3-dioxan-5-ylmethoxy)-2,3,5-trimethylpyridine 1-oxide

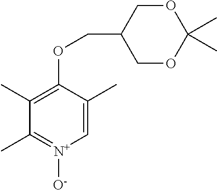

A mixture of 4-chloro-2,3,5-trimethylpyridine 1-oxide (840 g), (2,2-dimethyl-1,3-dioxan-5-yl)methanol (688 g), and toluene (2.52 L) was heated and refluxed while removing water. While continuing the azeotropic dehydration, potassium hydroxide (0.58 kg) was added thereto over 3 hours 45 minutes, and then the azeotropic dehydration was continued for a further 2.5 hours. The reaction system was then cooled to below 30° C., ethyl acetate (2.5 L) and 17% brine solution (3.5 L) were added thereto, and the reaction system was allowed to stand overnight. The ethyl acetate layer was separated off, and the aqueous layer was subjected to extraction with ethyl acetate (1.0 L×3). The ethyl acetate layers were combined, and filtered through Celite, and then vacuum concentration was carried out, whereby 1.20 kg of the target substance was obtained.

### (6) [4-(2,2-Dimethyl-1,3-dioxan-5-yl)methoxy-3,5-dimethylpyridin-2-yl]methanol monohydrate

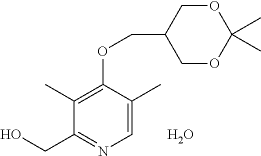

Acetic anhydride (1.10 kg) was added dropwise to a mixture of 4-(2,2-dimethyl-1,3-dioxan-5-ylmethoxy)-2,3,5-trimethylpyridine 1-oxide (1.20 kg) and sodium acetate (0.18 kg), which were heated to 50 to 60° C., over 1.5 hours. After 0.5 hours had elapsed, heating was carried out at 80° C. for 4.5 hours, and then the mixture was allowed to stand with the internal temperature being cooled to below 30° C., and then vacuum concentration was carried out. The resulting residue was dissolved in methanol (1.0 L), and was then added to a mixture of a 48% sodium hydroxide aqueous solution (0.71 kg) and cold water (2.85 L) over 1 hour. Stirring was carried out at room temperature for 5 hours 45 minutes, and then vacuum concentration was carried out. Water (3.0 L) was added to the concentrated residue, extraction was carried out with toluene (2.3 L×4), and then the toluene layers were combined and washed with water (1.2 L). The resulting organic layer was subjected to celite filtration, and then concentrated. Diisopropyl ether (1.15 L) was added at room temperature to the resulting residue, and then warm water (45° C., 74 mL) was further added. Once it had been confirmed that crystals had precipitated out, the mixture was stirred at 25° C. for 1 hour, and then added to heptane (3.6 L), and then the stirring was continued overnight. Stirring was carried out for a further 5 hours in a cooling-ice, and then filtration was carried out, whereby yellow crystals were obtained. Diisopropyl ether (3.5 L) was added to the resulting yellow crystals, and the crystals were dissolved at 50° C. Insoluble material was removed by filtration, and then gradual cooling was carried out, and maturation was carried out overnight at 5° C. The resulting crystals were filtered off, washed with heptane (0.5 L), and air-dried, whereby 0.69 kg of the target substance was obtained.

### (7) 2-[[[4-(2,2-Dimethyl-1,3-dioxan-5-yl)methoxy-3,5-dimethylpyridin-2-yl]methyl]thio]-1H-benzimidazole

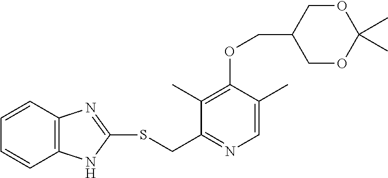

To [4-(2,2-dimethyl-1,3-dioxan-5-yl)methoxy-3,5-dimethylpyridin-2-yl]methanol monohydrate (690 g), and azeotropic dehydration was carried out (2.1 L×5, 1.75 L×1). Toluene (393 mL) was added to the resulting concentrated material, whereby 921 g of a toluene solution of [4-(2,2-dimethyl-1,3-dioxan-5-yl)methoxy-3,5-dimethylpyridin-2-yl]methanol was obtained.

Under nitrogen atmosphere, a solution of [4-(2,2-dimethyl-1,3-dioxan-5-yl)methoxy-3,5-dimethylpyridin-2-yl]methanol (845.7 g, percentage content 61.7%, amount contained 521.8 g, 1.855 mol) in toluene, tetrahydrofuran (2609 mL), toluene (669 mL), and triethylamine (375.3 g, 3.709 mol) were added thereto in this order, which was stirred in a cooling-dry ice/ethanol. Once 30 minutes had elapsed from the commencement of the cooling, methanesulfonyl chloride (254.9 g, 2.226 mol) was added thereto dropwise over 42 minutes. After the addition had been completed, stirring was carried out under cooling in an ice water bath. After approximately 1.5 hours, a solution of 2-mercaptobenzimidazole (334.28 g, 2.226 mol) in tetrahydrofuran (3653 mL) was added thereto over two minutes, and then the stirring was continued at room temperature for approximately 18 hours.

Toluene (3653 mL) was added to the reaction liquid, then a 20% w/w sodium hydroxide aqueous solution (1852.4 g) was added thereto, and then H₂O (2322 mL) was further added, and extraction and separation of the liquid layers were carried out. The organic layer was washed with a 20% w/w ammonium chloride aqueous solution (4174 g) twice, and was then further washed with H₂O (4147 mL).

The resulting organic layer was subjected to vacuum concentration (40° C.), whereby a brown oil was obtained (2.40 kg, containing 1446 mL of toluene and 168 mL of tetrahydrofuran, calculated from ¹H-NMR spectrum).

The resulting brown oil was transferred into a crystallization vessel, and washed down with toluene (119 mL), and stirring was carried out at room temperature. After 10 minutes, tert-butyl methyl ether (134 mL) was added thereto, and the stirring was continued at room temperature. After 20 minutes, tert-butyl methyl ether (127 mL) was further added thereto, and the stirring was continued at room temperature. After 30 minutes, tert-butyl methyl ether (266 mL) was further added dropwise over 20 minutes, and the stirring was continued at room temperature. After 1 minute, tert-butyl methyl ether (522 mL) was further added dropwise, whereupon crystals were seen to precipitate out after 8 minutes, the addition being completed over a total of 1 hour 20 minutes. After stirring at room temperature for 40 minutes, heptane (2348 mL) was added dropwise over 1 hour 17 minutes, and then stirring was carried out overnight at room temperature.

Approximately 15.5 hours after addition of heptane, the crystals precipitated out were filtered off by suction filtration, and rinsed with toluene/tert-butyl methyl ether/heptane (587 mL/391 mL/587 mL), and then subjected to suction drying. The resulting wet crystals were subjected to forced air drying (50° C.), whereby the target substance was obtained.

Yield: 619.0 g, percentage content: 96.5%, amount contained: 597.3 g, percentage yield: 77.8% (based on amount contained), HPLC purity: 98.0%<
<HPLC analysis conditions (reaction check, HPLC purity measurement, and quantification)>
Column: YMC-Pack Pro C18 AS-302 (5 μm, 4.6 mm×150 mm I.D.)
Eluent: A solution (MeCN/20 mM AcONH4 aq.=100/900 (v/v)), B solution (MeCN/20 mM AcONH4 aq.=800/200 (v/v))
Flow rate: 1.0 mL/min

### Detection: UV 254 nm

Oven temp.: 25° C.
Sample temp.: 25° C.
Gradient condition (time/B solution conc.): 0.01 min/0%→25 min/100%→30 min/100%→30.01 min/0%→40 min/stop

### RT=18.4 min

### (8) Crude sodium salt of optical isomer having short retention time of 2-[[[4-(2,2-dimethyl-1,3-dioxan-5-yl)methoxy-3,5-dimethylpyridin-2-yl]methyl]sulfinyl]-1H-benzimidazole

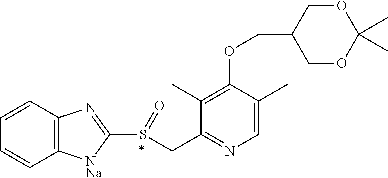

Under nitrogen atmosphere, 2-[[[4-(2,2-dimethyl-1,3-dioxan-5-yl)methoxy-3,5-dimethylpyridin-2-yl]methyl]thio]-1H-benzimidazole (580.3 g, percentage content 96.5%, amount contained 560.0 g, 1.354 mol), toluene (3864 mL), and H₂O (2.81 g, 0.156 mol) were added in this order, which was stirred was under heating to 60° C. After 6 minutes, L-(+)-diethyl tartrate (122.9 g, 0.596 mol) was added into the suspension, and then washing down with toluene (560 mL) was carried out. It was confirmed that dissolution had taken place after 30 minutes. After 8 minutes, titanium (IV) tetraisopropoxide (77.0 g, 0.271 mol) was added thereto, and then washing down with toluene (56 mL) was carried out, and stirring was carried out for approximately 1 hour with heating at the same temperature. Cooling to 8° C. was then changed to, N,N-diisopropylethylamine (56.01 g, 0.742 mol) was added thereto, and washing down with toluene (280 mL) was carried out. After 10 minutes, a toluene (840 ml) solution of cumene hydroperoxide (259.2 g, 1.422 mol) was added thereto over 47 minutes, and then after approximately 18.5 hours at 8° C. stirring was carried out. A cooled 30% w/w sodium thiosulfate aqueous solution (2240 g) was added thereto, stirring was carried out for 12 minutes, and then the aqueous layer was disposed of. A 4% w/w sodium hydroxide aqueous solution (2240 g) was added into the organic layer, stirring was carried out, and then after allowing to stand, the aqueous layer was separated off, whereby a sodium hydroxide aqueous extract of an optical isomer having a short retention time of 2-[[[4-(2,2-dimethyl-1,3-dioxan-5-yl)methoxy-3,5-dimethylpyridin-2-yl]methyl]thio]-1H-benzimidazole was obtained as a brownish yellow suspension. The sodium hydroxide aqueous extract (2.98 kg) of the optical isomer having a short retention time of 2-[[[4-(2,2-dimethyl-1,3-dioxan-5-yl)methoxy-3,5-dimethylpyridin-2-yl]methyl]thio]-1H-benzimidazole was added to toluene (7840 ml), and stirring was carried out. A20% w/w acetic acid aqueous solution (400 mL), an 8% NaOH aqueous solution (50 mL), and a 20% w/w acetic acid aqueous solution (8 mL) were added in this order into the mixture while stirring, so as to adjust the pH to 8.64, and then the mixture was allowed to stand, and the liquid layers were separated, the aqueous layer being disposed of. The organic layer was washed with a 5% w/w brine solution (2240 g), and the liquid layers were separated, whereby a toluene extract of the optical isomer having a short retention time of 2-[[[4-(2,2-dimethyl-1,3-dioxan-5-yl)methoxy-3,5-dimethylpyridin-2-yl]methyl]thio]-1H-benzimidazole (7.31 kg, content of optical isomer having short retention time of 2-[[[4-(2,2-dimethyl-1,3-dioxan-5-yl)methoxy-3,5-dimethylpyridin-2-yl]methyl]thio]-1H-benzimidazole 567.7 g, 1.322 mol) was obtained as a brownish yellow solution.

A28.3% sodium methoxide-methanol solution (245.6 g, 1.286 mol) was added into the toluene extract obtained over 1 minute while stirring at room temperature. Next, tert-butyl methyl ether (1120 mL) was added dropwise into the solution over 3 minutes, and stirring was carried out at room temperature, whereupon crystals were seen to precipitate out after 6 minutes, and then stirring was carried out for approximately 30 minutes as is. tert-Butyl methyl ether (7840 ml) was further added dropwise over 2 hours 40 minutes, and then stirring was continued overnight at room temperature.

Approximately 13 hours after adding tert-butyl methyl ether dropwise, the crystals precipitated out were filtered off by suction filtration, and rinsed with toluene/tert-butyl methyl ether (1047 mL/1193 mL), and then subjected to suction drying for 15 minutes. The resulting wet crystals were subjected to vacuum drying (40° C.), whereby the target substance was obtained.

Yield: 546.8 g, percentage content: 101.7%, amount contained: 546.8 g (assuming percentage content of 100%), percentage yield: 90.9% (based on yield amount),
HPLC purity: 98.2%, enantiomeric excess: 100% ee
<HPLC analysis conditions (reaction check, HPLC purity measurement, and quantification)>
Column: YMC-Pack Pro C18 AS-302 (5 μm, 4.6 mm×150 mm I.D.)
Eluent: A solution (MeCN/20 mM AcONH4aq.=100/900 (v/v)), B solution (MeCN/20 mM AcONH4aq.=800/200 (v/v))
Flow rate: 1.0 mL/min

### Detection: UV 254 nm

Oven temp.: 25° C.
Sample temp.: 25° C.
Gradient condition (time/B solution conc.): 0.01 min/0%→25 min/100%→30 min/100%→30.01 min/0%→40 min/stop

### RT=14.1 min

<HPLC analysis conditions (enantiomeric excess)
Column: Daicel Chiralpak IA (4.6 mm×250 mm I.D.)
Eluent: EtOH/MTBE=150/850 (v/v)
Flow rate: 1.0 mL/min

### Detection: UV 284 nm

Oven temp.: 25° C.
Sample temp.: 25° C.

### (9) Purified sodium salt of optical isomer having short retention time of 2-[[[4-(2,2-dimethyl-1,3-dioxan-5-yl)methoxy-3,5-dimethylpyridin-2-yl]methyl]sulfinyl]-1H-benzimidazole

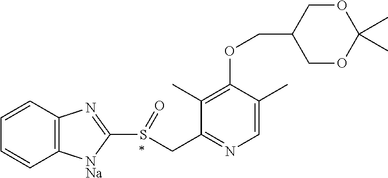

Ethanol (1074 mL) was added to the crude sodium salt of the optical isomer having a short retention time of 2-[[[4-(2,2-dimethyl-1,3-dioxan-5-yl)methoxy-3,5-dimethylpyridin-2-yl]methyl]sulfinyl]-1H-benzimidazole (536.8 g, 1.189 mol), and dissolution was carried out at room temperature, and then tert-butyl methyl ether (1074 mL) was further added thereto. The resulting solution was subjected to suction filtration with a Hyflo Super-Cel bed (107.4 g, washed in order with ethanol/tert-butyl methyl ether (1074 mL/1074 mL) and tert-butyl methyl ether (537 mL)), and rinsed with ethanol/tert-butyl methyl ether (215 mL/215 mL).

The resulting filtrate was transferred into a crystallization vessel, and washed down with ethanol/tert-butyl methyl ether (54 mL/54 mL), and then stirring was commenced at room temperature. tert-Butyl methyl ether (1610 mL) was added dropwise over 6 minutes, and the stirring was continued at room temperature. After 11 minutes, tert-butyl methyl ether (268 mL) was added dropwise over 2 minutes, and the stirring was continued, precipitation of crystals being seen after 1 minute. Stirring was carried out at room temperature as is for 31 minutes, and then tert-butyl methyl ether (268 mL) was added dropwise over 9 minutes. Stirring was carried out for 8 minutes at room temperature, and then tert-butyl methyl ether (8589 mL) was further added dropwise over 1 hour and 10 minutes, and the stirring was continued at room temperature.

Approximately 22 hours after addition of tert-butyl methyl ether had been completed, the precipitated crystals were filtered off by suction filtration while spraying in nitrogen, and washed with ethanol/tert-butyl methyl ether (107 mL/966 mL) and tert-butyl methyl ether (1074 mL) in this order, and then subjected to suction drying for 8 minutes. Of the resulting wet crystals (584.54 g), 531.10 g was subjected to vacuum drying (50° C.), whereby the target substance was obtained.

Yield: 419.6 g, HPLC purity: 99.4%<
HPLC analysis conditions (HPLC purity measurement, and quantification)>
Column: YMC-Pack Pro C18 AS-302 (5 μm, 4.6 mm×150 mm I.D.)
Eluent: A solution (MeCN/20 mM AcONH4aq.=100/900 (v/v)), B solution (MeCN/20 mM AcONH4aq.=800/200 (v/v))
Flow rate: 1.0 mL/min

### Detection: UV 254 nm

Oven temp.: 25° C.
Sample temp.: 25° C.
Gradient condition (time/B solution conc.): 0.01 min/0%→25 min/100%→30 min/100%→30.01 min/0%→40 min/stop

### RT=14.1 min

### EXAMPLES

### Example 1

### Sodium Salt of 2-[[[4-[(2,2-dimethyl-1,3-dioxan-5-yl)methoxy]-3,5-dimethylpyridin-2-yl]methyl]sulfinyl]-1H-benzimidazole

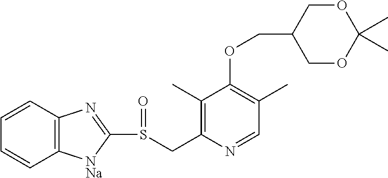

### (1a) 5,9-dioxaspiro[3.5]non-7-ylmethanol

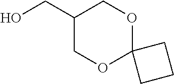

Cyclobutanone (3.3 g, 47.1 mmol), 2-(hydroxymethyl)-1,3-propanediol (5 g, 47.1 mmol), p-toluenesulfonic acid monohydrate (450 mg, 2.37 mmol), and benzene (50 ml) were mixed together in a round-bottom flask equipped with a Dean-Stark and a cooling tube, and the mixture was stirred for 6 hours under reflux, and then allowed to stand at room temperature overnight. Triethylamine (2 ml) was added to the reaction mixture, and then the reaction liquid was concentrated. The resulting residue was purified by silica gel column chromatography (silica gel, eluent: heptane/ethyl acetate=1/1-1/3 gradient), whereby the title compound (5.56 g, yield 67.1%) was obtained as a colorless oil.

¹H-NMR (400 MHz, DMSO-d₆) δ ppm: 1.56-1.66 (2H, m), 1.66-1.76 (1H, m), 2.05-2.15 (4H, m), 3.33 (2H, dd, J=5, 6 Hz), 3.50 (2H, dd, J=8, 12 Hz), 3.76 (2H, dd, J=4, 12 Hz), 4.54 (1H, t, J=5 Hz).

### (1b) 4-(5,9-Dioxaspiro[3.5]non-7-ylmethoxy)-2,3,5-trimethylpyridine 1-oxide

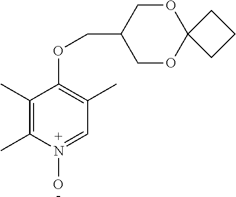

To a mixture of 4-chloro-2,3,5-trimethylpyridine 1-oxide (5.48 g, 31.9 mmol), the 5,9-dioxaspiro[3.5]non-7-ylmethanol (5.56 g, 35.1 mmol) and toluene (20 ml) was added potassium hydroxide (3.94 g, 70.2 mmol) in a round-bottom flask equipped with a Dean-Stark and a cooling tube. The reaction mixture was stirred for 6 hours while withdrawing water under heating and reflux, and then allowed to stand at room temperature overnight. Ethyl acetate was then added to the reaction mixture, NH silica gel was added and concentration and drying were carried out, and then purification was carried out by silica gel column chromatography (NH silica gel), whereby the title compound (4.42 g, yield 47.2%) was obtained as a yellow viscous substance.

¹H-NMR (400 MHz, DMSO-d₆) δ ppm: 1.58-1.68 (2H, m), 2.07-2.20 (5H, m), 2.11 (3H, s), 2.14 (3H, s), 2.29 (3H, s), 3.73 (2H, dd, J=6, 12 Hz), 3.77 (2H, d, J=7 Hz), 3.93 (2H, dd, J=4, 12 Hz), 8.04 (1H, s).

### (1c) [4-(5,9-Dioxaspiro[3.5]non-7-ylmethoxy)-3,5-dimethylpyridin-2-yl]methyl acetate

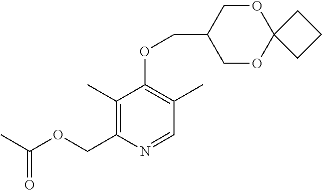

A mixture of 4-(5,9-dioxaspiro[3.5]non-7-ylmethoxy)-2,3,5-trimethylpyridine 1-oxide obtained in (1b) above (3.22 g, 11 mmol) and acetic anhydride (30 ml) was stirred at 85° C. for 1 hour. The reaction mixture was then concentrated using a vacuum pump. The resulting residue was purified by silica gel column chromatography (silica gel, eluent: heptane/ethyl acetate=1/1-0/1 gradient), whereby the title compound (2.95 g, yield 80.0%) was obtained as a yellow oil.

¹H-NMR (400 MHz, DMSO-d₆) δ ppm: 1.58-1.69 (2H, m), 2.03 (3H, s), 2.06-2.23 (5H, m), 2.16 (3H, s), 2.18 (3H, s), 3.74 (2H, dd, J=6, 11 Hz), 3.83 (2H, d, J=7 Hz), 3.94 (2H, dd, J=4, 11 Hz), 5.09 (2H, s), 8.17 (1H, s).

### (1d) [4-(5,9-Dioxaspiro[3.5]non-7-ylmethoxy)-3,5-dimethylpyridin-2-yl]methanol

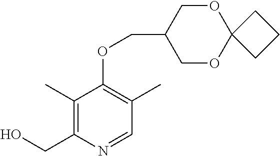

To a mixture of [4-(5,9-dioxaspiro[3.5]non-7-ylmethoxy)-3,5-dimethylpyridin-2-yl]methyl acetate obtained in (1c) above (1.75 g, 5.22 mmol) and methanol (15 ml) was added a 5N sodium hydroxide aqueous solution (10 ml), which was stirred at room temperature for 2 hours. To the reaction mixture was added a saturated ammonium chloride aqueous solution (10 ml) such that a pH of the solution became approximately 10, and then concentration was carried out. Ethyl acetate was added to the resulting residue, thorough stirring was carried out, and then the organic layer was separated off. The organic layer was washed with brine, dried over anhydrous sodium sulfate, and then concentrated, whereby the title compound (1.54 g, quantitative yield) was obtained as a yellow oil.

¹H-NMR (400 MHz, DMSO-d₆) δ ppm: 1.58-1.68 (2H, m), 2.07-2.21 (5H, m), 2.17 (6H, s), 3.74 (2H, dd, J=6, 12 Hz), 3.81 (2H, d, J=7 Hz), 3.94 (2H, dd, J=4, 12 Hz), 4.49 (2H, d, J=5 Hz), 4.95 (1H, t, J=5 Hz), 8.14 (1H, s)

### (1e) 2-[[[4-(5,9-Dioxaspiro[3.5]non-7-ylmethoxy)-3,5-dimethylpyridin-2-yl]methyl]thio]-1H-benzimidazole

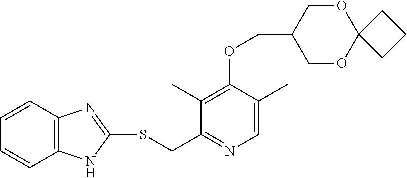

To a mixture of [4-(5,9-dioxaspiro[3.5]non-7-ylmethoxy)-3,5-dimethylpyridin-2-yl]methanol obtained in (1d) above (297 mg, 1.01 mmol) and tetrahydrofuran (7 ml) was added triethylamine (0.29 ml, 2.08 mmol). The reaction mixture was cooled in an ice bath, methanesulfonyl chloride (0.12 ml, 1.55 mmol) was added dropwise thereto, and stirring was carried out for 30 minutes while cooling in the ice bath. To the reaction mixture were added a saturated sodium hydrogencarbonate aqueous solution and ethyl acetate, which was stirred, and then the organic layer was separated off. The organic layer was washed with water and brine, dried over anhydrous sodium sulfate, and then concentrated, and then solvent was further evaporated off using a vacuum pump. Ethanol (7 ml) was added to the resulting residue, and 2-mercaptobenzimidazole (152 mg, 1.01 mmol) and sodium hydroxide (162 mg, 4.04 mmol) were added thereto in this order, and stirring was carried out at room temperature for 15 hours. The reaction mixture was concentrated, and then NH silica gel was added and concentration and drying were carried out, and then purification was carried out by silica gel column chromatography (silica gel, eluent: heptane/ethyl acetate=1/1-0/1 gradient), whereby the title compound (370 mg, yield 86.1%) was obtained as a white foam.

¹H-NMR (400 MHz, DMSO-d₆) δ ppm: 1.58-1.69 (2H, m), 2.07-2.20 (5H, m), 2.17 (3H, s), 2.26 (3H, s), 3.74 (2H, dd, J=6, 12 Hz), 3.82 (2H, d, J=7 Hz), 3.93 (2H, dd, J=4, 12 Hz), 4.66 (2H, s), 7.07-7.14 (2H, m), 7.39-7.47 (2H, m), 8.16 (1H, s)

### (1f) 2-[[[2-[(1H-benzimidazol-2-ylthio)methyl]-3,5-dimethylpyridin-4-yl]oxy]methyl]propane-1,3-diol

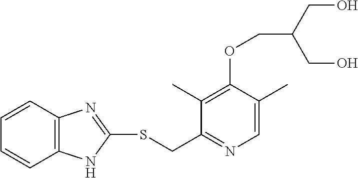

To a mixture of 2-[[[4-(5,9-dioxaspiro[3.5]non-7-ylmethoxy)-3,5-dimethylpyridin-2-yl]methyl]thio]-1H-benzimidazole obtained in (1e) above (80 mg, 0.188 mmol) and methanol (0.5 ml) was added 2N hydrochloric acid (0.5 ml), which was stirred at room temperature for 1.5 hours. To the reaction mixture was added a 2N sodium hydroxide aqueous solution (0.5 ml), which was stirred was for approximately 30 minutes. The precipitate in the mixture was filtered off, and was washed with water. Methanol was added to the resulting precipitate, and solvent was evaporated off (under a reduced pressure, approximately 40° C.)₇ and then solvent was further evaporated off using a vacuum pump, whereby the title compound (50 mg, yield 71.2%) was obtained as a white solid.

¹H-NMR (400 MHz, DMSO-d₆) δ ppm: 1.93-2.03 (1H, m), 2.18 (3H, s), 2.27 (3H, s), 3.50-3.61 (4H, m), 3.82 (2H, d, J=6 Hz), 4.53 (2H, brs), 4.66 (2H, s), 7.07-7.14 (2H, m), 7.38-7.48 (2H, m), 8.14 (1H, s)

### (1 g) 2-[[[4-[(2,2-Dimethyl-1,3-dioxan-5-yl)methoxy]-3,5-dimethylpyridin-2-yl]methyl]thio]-1H-benzimidazole

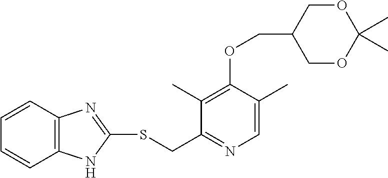

### (Method 1)

To a mixture of 2-[[[2-[(1H-benzimidazol-2-ylthio)methyl]-3,5-dimethylpyridin-4-yl]oxy]methyl]propane-1,3-diol (100 mg, 0.268 mmol) and N,N-dimethylformamide (2 ml) was added 2,2-diethoxypropane (0.09 ml, 0.565 mmol), which was stirred for 2 hours at 120° C. To the reaction mixture were added water and ethyl acetate, which was stirred, and then the organic layer was separated off. The organic layer was washed with water and brine, dried over anhydrous sodium sulfate, and then concentrated. The resulting residue was purified by silica gel column chromatography (silica gel, eluent: heptane/ethyl acetate=1/1-0/1 gradient), whereby the title compound (48 mg, percentage content 95%, yield 41.1%) was obtained as a colorless oil.

¹H-NMR (400 MHz, DMSO-d₆) δ ppm: 1.31 (3H, s), 1.34 (3H, s), 2.03-2.13 (1H, m), 2.18 (3H, s), 2.26 (3H, s), 3.79 (2H, dd, J=6, 12 Hz), 3.85 (2H, d, J=7 Hz), 3.99 (2H, dd, J=4, 12 Hz), 4.67 (2H, s), 7.06-7.13 (2H, m), 7.39-7.47 (2H, m), 8.16 (1H, s)

### (Method 2)

To a mixture of 2-[[[2-[(1H-benzimidazol-2-ylthio)methyl]-3,5-dimethylpyridin-4-yl]oxy]methyl]propane-1,3-diol (300 mg, 0.803 mmol) and acetone (12 ml), which was cooled in an ice bath, was added iron (III) chloride (130 mg, 0.803 mmol) followed by stirring for 4 hours while cooling in the ice bath. Iron (III) chloride (130 mg, 0.803 mmol) was further added to the reaction mixture followed by stirring at room temperature for 18 hours. To the reaction mixture were added saturated sodium hydrogencarbonate and ethyl acetate, which was stirred, and then the organic layer was extracted. The organic layer was dried over anhydrous sodium sulfate, and then concentrated. The resulting residue was purified by silica gel column chromatography (silica gel, eluent: heptane/ethyl acetate=1/1-0/1 gradient), whereby the title compound (180 mg, yield 54.2%) was obtained as a pale yellow foam. It was confirmed from ¹H-NMR (400 MHz, DMSO-d₆) measurement that this compound was the same as the compound obtained through method 1.

### (1 h) 2-[[[4-[(2,2-Dimethyl-1,3-dioxan-5-yl)methoxy]-3,5-dimethylpyridin-2-yl]methyl]sulfinyl]-1H-benzimidazole

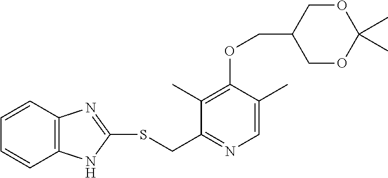

Under a nitrogen atmosphere, to a mixture of 2-[[[4-[(2,2-dimethyl-1,3-dioxan-5-yl)methoxy]-3,5-dimethylpyridin-2-yl]methyl]thio]-1H-benzimidazole (424 mg, 1.03 mmol), toluene (20 ml) and methanol (2 ml) was added a mixture of 3-chloroperbenzoic acid (246 mg, 0.927 mmol assuming 65% content), toluene (1 ml) and methanol (1 ml) dropwise over 5 minutes at −65° C., the resulting mixture was stirred at −65° C. for 45 minutes. To the reaction mixture were added a saturated sodium hydrogencarbonate aqueous solution and ethyl acetate were added, which was stirred, and then the organic layer was separated off. The organic layer was dried over anhydrous sodium sulfate, and then concentrated. The residue was purified by silica gel column chromatography (NH silica gel: 20 g, eluent: dichloromethane, dichloromethane/methanol=10/1). Diethyl ether was then added to the residue. Precipitate produced in the mixture was filtered off, and was washed with diethyl ether, whereby the title compound (274 mg, yield 61.9%) was obtained as a white solid.

¹H-NMR (400 MHz, DMSO-d₆) δ ppm: 1.32 (3H, s), 1.36 (3H, s), 2.02-2.13 (1H, m), 2.16 (3H, s), 2.20 (3H, s), 3.74-3.84 (4H, m), 4.00 (2H, dd, J=4, 12 Hz), 4.70 (1H, d, J=14 Hz), 4.79 (1H, d, J=14 Hz), 7.26-7.33 (2H, m), 7.60-7.70 (2H, m), 8.18 (1H, s)

### (1i) Sodium salt of 2-[[[4-[(2,2-dimethyl-1,3-dioxan-5-yl)methoxy]-3,5-dimethylpyridin-2-yl]methyl]sulfinyl]-1H-benzimidazole

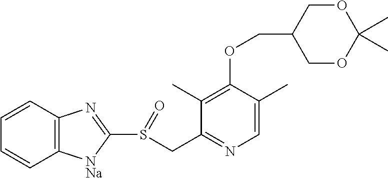

To a mixture of 2-[[[4-[(2,2-dimethyl-1,3-dioxan-5-yl)methoxy]-3,5-dimethylpyridin-2-yl]methyl]sulfinyl]-1H-benzimidazole obtained in (1 h) above (274 mg, 0.638 mmol) and ethanol (10 ml) was added a 1N sodium hydroxide aqueous solution (635 μl, 0.638 mmol assuming 1.004 M concentration) at room temperature, and the resulting mixture was concentrated. Ethanol was added to the residue, and solvent was evaporated off, and then ethanol was again added to the residue, and solvent was evaporated off. Diethyl ether was added to the residue, sonication was carried out with ultrasound, and then concentration was carried out, whereby the title compound (260 mg, yield 90.3%) was obtained as a white solid.

¹H-NMR (400 MHz, DMSO-d₆) δ ppm: 1.33 (3H, s), 1.36 (3H, s), 2.03-2.14 (1H, m), 2.20 (3H, s), 2.21 (3H, s), 3.76-3.87 (4H, m), 4.00 (2H, dd, J=4, 11 Hz), 4.39 (1H, d, J=13 Hz), 4.75 (1H, d, J=13 Hz), 6.81-6.91 (2H, m), 7.40-7.48 (2H, m), 8.23 (1H, s)

### (1j) 2-[[[2-(Hydroxymethyl)-3,5-dimethylpyridin-4-yl]oxy]methyl]propane-1,3-diol

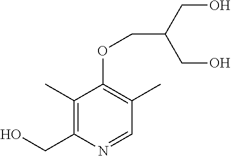

To a mixture of [4-(5,9-dioxaspiro[3.5]non-7-ylmethoxy)-3,5-dimethylpyridin-2-yl]methanol obtained in (1d) above (200 mg, 0.682 mmol) and methanol (2 ml) was added 2N hydrochloric acid (2 ml), which was stirred at room temperature for 50 minutes. To the reaction mixture was added a 2N sodium hydroxide aqueous solution such that the liquid became substantially neutral. Methanol was added to the mixture, and solvent was evaporated off (under a reduced pressure, approximately 40° C.), and then methanol was again added to the residue, and solvent was evaporated off (under a reduced pressure, approximately 40° C.). Methanol was added to the resulting residue, and then NH silica gel was added and concentration and drying were carried out. The solid obtained was purified by silica gel column chromatography (NH silica gel, ethyl acetate/methanol=1/0-4/1 gradient), whereby the title compound (135 mg, yield 82%) was obtained as a white solid.

¹H-NMR (400 MHz, DMSO-d₆) δ ppm: 1.91-2.03 (1H, m), 2.17 (6H, s), 3.56 (4H, dd, J=5, 6 Hz), 3.81 (2H, d, J=6 Hz), 4.49 (2H, d, J=5 Hz), 4.52 (2H, t, J=5 Hz), 4.94 (1H, t, J=5 Hz), 8.13 (1H, s)

### (1 k) [4-[(2,2-Dimethyl-1,3-dioxan-5-yl)methoxy]-3,5-dimethylpyridin-2-yl]methanol

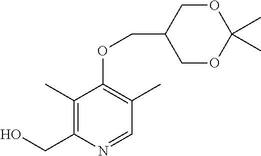

To a mixture of 2-[[[2-(hydroxymethyl)-3,5-dimethylpyridin-4-yl]oxy]methyl]propane-1,3-diol obtained in (1j) above (118 mg, 0.489 mmol) and acetone (5 ml) was added iron chloride(III) (238 mg, 1.47 mmol), which was stirred at 50° C. for 2 hours. The reaction mixture was cooled in an ice bath, and ethyl acetate (10 ml) and a 2N NaOH aqueous solution (5 ml) were added thereto followed by stirring for 15 minutes. The reaction mixture was filtered through Celite, and then the celite was washed with ethyl acetate. The filtrates were combined and thoroughly stirred, and then the organic layer was separated off. The organic layer was washed with brine, dried over anhydrous sodium sulfate, and then concentrated. The resulting residue was purified by silica gel column chromatography (NH silica gel, eluent: heptane/ethyl acetate=1/1-0/1), whereby the title compound (39 mg, 28.3% yield) was obtained as a colorless oil.

¹H-NMR (400 MHz, DMSO-d₆) δ ppm: 1.31 (3H, s), 1.35 (3H, s), 2.03-2.13 (1H, m), 2.18 (6H, s), 3.80 (2H, dd, J=6, 12 Hz), 3.84 (2H, d, J=7 Hz), 4.00 (2H, dd, J=4, 12 Hz), 4.49 (2H, d, J=5 Hz), 4.96 (1H, t, J=5 Hz), 8.14 (1H, s)

### (1l) 2-[[[4-[(2,2-Dimethyl-1,3-dioxan-5-yl)methoxy]-3,5-dimethylpyridin-2-yl]methyl]thio]-1H-benzimidazole

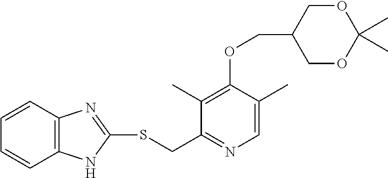

Under nitrogen atmosphere, to a mixture of [4-[(2,2-dimethyl-1,3-dioxan-5-yl)methoxy]-3,5-dimethylpyridin-2-yl]methanol (504 mg, 1.79 mmol), triethylamine (500 μl, 3.58 mmol) and tetrahydrofuran (15 ml) was added methanesulfonyl chloride (208 μl, 2.69 mmol) dropwise over 15 minutes at from 1 to 3° C., and the resulting mixture was stirred at from 1 to 3° C. for 1 hour and 25 minutes. To the reaction mixture was added 2-mercaptobenzimidazole (271 mg, 1.8 mmol), which was stirred was for 64 hours and 20 minutes. To the reaction mixture were added ethyl acetate and a saturated sodium hydrogencarbonate aqueous solution, which was stirred thoroughly, and then the organic layer was separated off. The organic layer was dried over anhydrous magnesium sulfate, and then concentrated. The residue was purified by silica gel column chromatography (silica gel: 30 g, eluent: heptane I ethyl acetate=42/58, 22/78, ethyl acetate), whereby the title compound (442 mg, yield 59.7%) was obtained as a colorless foam.

¹H-NMR (400 MHz, DMSO-d₆) δ ppm: 1.33 (3H, s), 1.36 (3H, s), 2.05-2.16 (1H, m), 2.20 (3H, s), 2.28 (3H, s), 3.81 (2H, dd, J=6, 12 Hz), 3.87 (2H, d, J=7 Hz), 4.02 (2H, dd, J=4, 12 Hz), 4.69 (2H, s), 7.09-7.16 (2H, m), 7.41-7.50 (2H, m), 8.18 (1H, s)

### Example 2

### 2-[[[4-[(2,2-Dimethyl-1,3-dioxan-5-yl)methoxy]-3,5-dimethylpyridin-2-yl]methyl]thio]-1H-benzimidazole

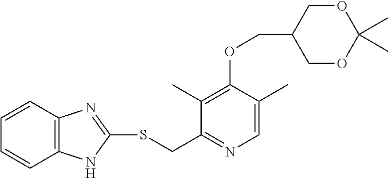

### (2a) 2-[[[4-Chloro-3,5-dimethylpyridin-2-yl]methyl]thio]-1H-benzimidazole

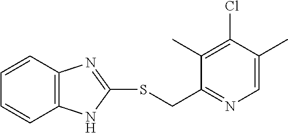

To a mixture of 4-chloro-2,3,5-trimethylpyridine 1-oxide (6.34 g, 36.9 mmol), lithium chloride (3.13 g, 73.8 mmol) and toluene (60 ml) was added methanesulfonyl chloride (8.57 ml, 111 mmol) at from 0 to 3° C. (internal temperature) with stirring and cooling in an ice bath. The reaction mixture was stirred at room temperature for 13 hours and 20 minutes, then heating and refluxing were carried out for 10 hours, and then the mixture was further allowed to stand at room temperature for 40 days and 10 hours. A saturated sodium hydrogencarbonate aqueous solution was then added into the reaction mixture dropwise in a cooling-ice. Ethyl acetate was added to the reaction mixture, followed by stirring thoroughly, and then the organic layer was separated off. Ethyl acetate was added to the aqueous layer followed by stirring thoroughly, and then the organic layer was separated off. The organic layers were combined and dried over anhydrous magnesium sulfate, then filtered through a silica gel pad, and then subjected to vacuum concentration. To the resulting residue were added 2-mercaptobenzimidazole (5.54 g, 36.9 mmol), a mixture of methanol and tetrahydrofuran (50 ml/50 ml), and triethylamine (5.14 ml, 36.9 mmol) at 0° C. in this order. The reaction mixture was stirred at room temperature for 4 hours, and then subjected to vacuum concentration. Methanol (50 ml) was added to the residue followed by stirring, and then precipitate in the mixture was filtered off, and the precipitate was washed with methanol (30 ml) twice and then dried, whereby the title compound (4.2 g, yield 37.5%) was obtained as a white solid.

¹H-NMR (400 MHz, DMSO-d₆) δ ppm: 2.29 (3H, s), 2.44 (3H, s), 4.78-4.88 (2H, brs), 7.16-7.31 (2H, m), 7.46-7.58 (2H, m), 8.28 (1H, s)

### (2b) 2-[[[4-[(2,2-Dimethyl-1,3-dioxan-5-yl)methoxy]-3,5-dimethylpyridin-2-yl]methyl]thio]-1H-benzimidazole

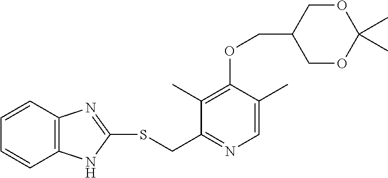

A mixture of 2-[[[4-chloro-3,5-dimethylpyridin-2-yl]methyl]thio]-1H-benzimidazole obtained in (2a) above (110 mg, 0.342 mmol), (2,2-dimethyl-1,3-dioxan-5-yl)methanol (100 mg, 0.684 ml), potassium t-butoxide (92.1 mg, 0.821 mmol) and dimethyl sulfoxide (0.6 ml) was stirred at 60° C. (external temperature) for 2 hours, which was then further stirred at 90° C. (external temperature) for 67 hours. To the reaction mixture were added ethyl acetate (5 ml) and then water (1 ml). After stirring thoroughly, the organic layer was separated off. The organic layer was dried over anhydrous magnesium sulfate, and subjected to vacuum concentration. The residue was purified by silica gel column chromatography (eluent: AcOEt/heptane=1/1-1/0), whereby the title compound (36 mg, yield 25.5%) was obtained as a colorless oil.

¹H-NMR (400 MHz, DMSO-d₆) δ ppm: 1.31 (3H, s), 1.34 (3H, s), 2.03-2.14 (1H, m), 2.18 (3H, s), 2.26 (3H, s), 3.79 (2H, dd, J=6, 12 Hz), 3.85 (2H, d, J=7 Hz), 4.00 (2H, dd, J=4, 12 Hz), 4.67 (2H, s), 7.07-7.14 (2H, m), 7.39-7.48 (2H, m), 8.16 (1H, s)

### Test Example 1

### Gastric Acid Secretion Inhibiting Effect in Dogs Having Chronic Gastric Fistula

### (1) Method

Using large dogs (body weight approximately 14 to 19 kg) having a chronic gastric fistula, the gastric acid secretion inhibiting action and gastric acid secretion inhibiting action persistence of test compounds of the Examples was investigated. The experiment was carried out over 2 days. On the first day, gastric juice was collected every 20 minutes under conditions of intravenously administering histamine (50 or 75 μg/kg/h) continuously for 3 hours. 1 Hour after commencing the histamine administration, the test compound suspended or dissolved in a 0.5% methylcellulose solution was administered at a volume of 0.1 ml/kg via an indwelling catheter in the duodenum. The gastric acid secretion inhibiting action of the test compound was then investigated over the subsequent 2 hours. On the second day (24 hours after the administration of the test compound), gastric juice was collected every 20 minutes under conditions of intravenously administering histamine continuously for 2 hours, and the gastric acid secretion inhibiting action persistence was investigated. After measuring the amount of gastric juice, a 0.5 ml sample of the gastric juice was taken, and neutralization titration was carried out to a pH of 7.0 with a 0.04 mol/l sodium hydroxide solution so as to measure the acid concentration. The acid concentration was multiplied by the amount of gastric juice so as to determine the gastric acid output. The gastric acid secretion inhibiting action was evaluated as the gastric acid secretion suppression rate (%) on the first day. The gastric acid secretion inhibiting action (%) was calculated using the following formula. In the case of 2 or more dogs, the mean over all of the dogs was calculated and is shown.

Gastric acid secretion inhibiting action (%)=(A−B)/A×100

[A] Gastric acid output over 20 minutes from 40 minutes to 1 hour after commencement of histamine administration
[B] Gastric acid output over 20 minutes from 1 hour 40 minutes to 2 hours after administration of test compound

The gastric acid secretion inhibiting action persistence was evaluated as the gastric acid secretion suppression rate (%) on the second day. The gastric acid secretion inhibiting action persistence (%) was calculated using the following formula.

Gastric acid secretion inhibiting action persistence (%)=(C−D)/C×100

[C] Total gastric acid output from commencement of histamine administration on first day to 1 hour thereafter [D] Total gastric acid output from commencement of histamine administration on second day to 1 hour thereafter

### (2) Results

Table 1 shows the results of the gastric acid secretion inhibiting effects for the dogs having the chronic gastric fistula.

TABLE 1

| | | | Gastric Acid | Gastric Acid |
|---|---|---|---|---|
| | Dose | | Secretion | Secretion |
| | Administered | Number | Inhibiting | Inhibiting |
| | (mg/kg, i.d.) | of Dogs | Action (%) | Persistence (%) |
| Compound X | 0.1 | 4 | 8 | 6 |
| Compound X | 0.2 | 4 | 65 | 46 |
| Compound X | 0.4 | 10 | 97 | 77 |
| Compound X | 0.8 | 8 | 100 | 89 |

Compound X: Sodium Salt of Optical Isomer Having Short Retention Time of 2-[[[4-(2,2-dimethyl-1,3-dioxan-5-yl)methoxy-3,5-dimethylpyridin-2-yl]methyl]sulfinyl]-1H-benzimidazole

From the results shown in Table 1, it was ascertained that above compound X has a good gastric acid secretion inhibiting action and gastric acid secretion inhibiting persistence.

### INDUSTRIAL APPLICABILITY

The production process according to the present invention can be used for producing on an industrial scale compound (1) or a salt thereof which is useful as a compound having an excellent gastric acid secretion inhibiting action.

1. A process for the production of a compound (1) represented by the formula or a salt thereof 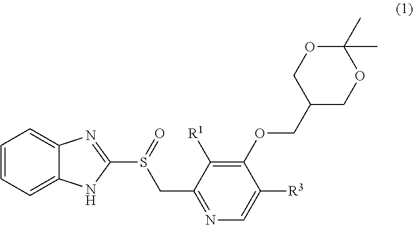
(wherein R1 and R3 independently represent a hydrogen atom or a methyl group), the process comprising the steps of:
(a) reacting together a compound (3T) represented by the formula 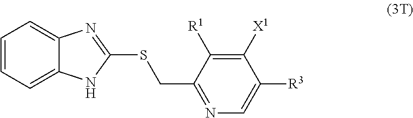
(wherein XI represents a leaving group, and R1 and R3 are defined as above) and (2,2-dimethyl-1,3-dioxan-5-yl)methanol represented by the formula or a hydrate thereof, 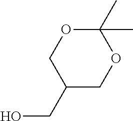
so as to produce a compound (2T) represented by the formula 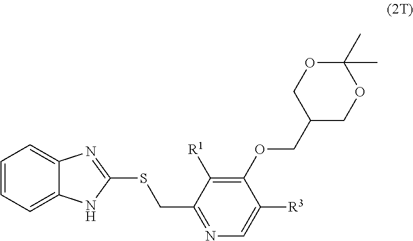
(wherein R1 and R3 are defined as above); and
(b) reacting the compound represented by the formula (2T) with an oxidizing agent.

2. A process for the production of a compound (1) represented by the formula or a salt thereof 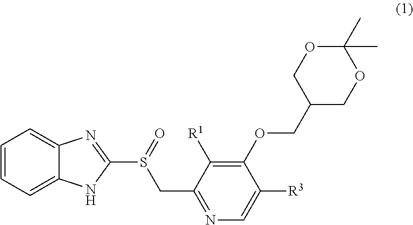
(wherein R1 and R3 independently represent a hydrogen atom or a methyl group), the process comprising the steps of:
(c) subjecting a compound (3U) represented by the formula 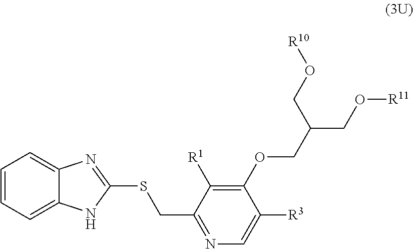
(wherein R10 and R11 independently represent a hydrogen atom or a protecting group of a hydroxyl group, or R10 and R11 are combined so as to represent a methylene group (the methylene group optionally having thereon one or two groups selected from a C1-C6 alkyl group, a C1-C6 alkoxy group, a phenyl group optionally having thereon one or two methoxy groups, and trichloromethyl groups), a carbonyl group, a 1,1-cyclopropylene group, a 1,1-cyclobutylene group, a 1,1-cyclopentylene group, or a 1,1-cyclohexylene group (provided that R10 and R11 are combined so as to represent a 2,2-propylene group being excluded), and R1 and R3 are defined as above)
to a deprotection reaction, and then reacting with an acetalizing reagent, so as to produce a compound (2T) represented by the formula 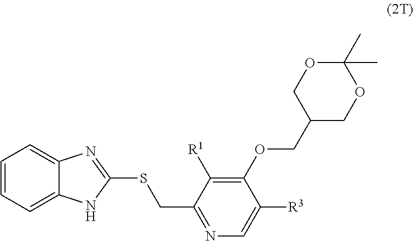
(wherein R1 and R3 are defined as above); and
(b) reacting the compound represented by the formula (2T) with an oxidizing agent.

3. A process for the production of a compound (1Z) represented by the formula or a salt thereof 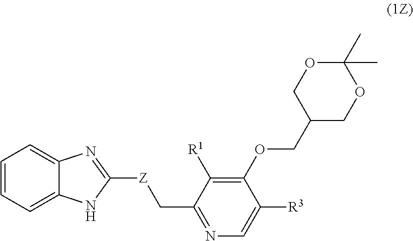
(wherein Z represents —S— or —SO—, and R1 and R3 independently represent a hydrogen atom or a methyl group),
the process comprising:
reacting together a compound (2Z) represented by the formula 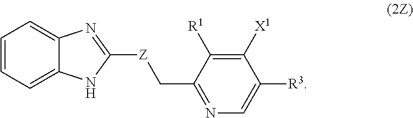
(wherein X1 represents a leaving group, and Z, R1 and R3 are defined as above) and (2,2-dimethyl-1,3-dioxan-5-yl)methanol represented by the formula

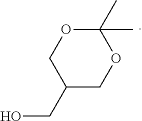

4. A process for the production of a compound (1Z) represented by the formula or a salt thereof 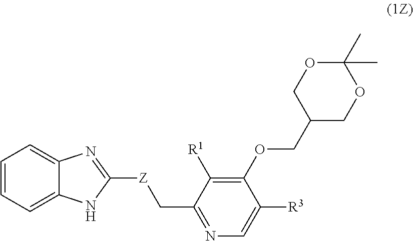
(wherein Z, R1 and R3 are defined as above),
the process comprising the steps of:
subjecting a compound (3Z) represented by the formula 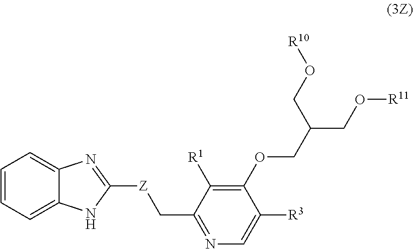
(wherein Z represents —S— or —SO—, and R10 and R11 independently represent a hydrogen atom or a protecting group of a hydroxyl group, or R10 and R11 are combined so as to represent a methylene group (the methylene group optionally having thereon one or two groups selected from a C1-C6 alkyl group, a C1-C6 alkoxy group, a phenyl group optionally having thereon one or two methoxy groups, and trichloromethyl groups), a carbonyl group, a 1,1-cyclopropylene group, a 1,1-cyclobutylene group, a 1,1-cyclopentylene group, or a 1,1-cyclohexylene group (provided that R10 and R11 are combined so as to represent a 2,2-propylene group being excluded))
to a deprotection reaction,
and then reacting with an acetalizing reagent.
